(19) United States
(12) Patent Application Publication
(10) Pub. No.: US 2018/0133238 A1
(43) Pub. Date: May 17, 2018

(54) ORAL ADMINISTRATION OF ANTHOCYANINS, ANTHOCYANIDINS, AND THEIR METABOLITES FOR THE TREATMENT OF INFLAMMATION IN AN INFLAMMED JOINT

(71) Applicant: Lanny Leo Johnson, Frankfort, MI (US)

(72) Inventor: Lanny Leo Johnson, Frankfort, MI (US)

(21) Appl. No.: 15/839,491

(22) Filed: Dec. 12, 2017

Related U.S. Application Data
(63) Continuation of application No. 14/533,820, filed on Nov. 5, 2014, now abandoned.

(51) Int. Cl. A61K 31/7048; A61K 31/192

(52) U.S. Cl. CPC A61K 31/7048; A61K 31/192

(57) ABSTRACT

The invention provides methods of reducing inflammation and improving the catabolic/anabolic state to promote healing of the cartilage in a joint by providing an oral dosage of either an anthocyanin or an anthocyanidin or their metabolites or a combination thereof. The treatment can occur before surgery, injury or inflammation occurs as a prophylactic treatment and/or can continue during or after the surgery, injury or inflammation has begun.

### REFERENCE TO EARLIER FILED APPLICATION

This application is a continuation of U.S. patent application Ser. No. 14/533,820, filed Nov. 4, 2014, the disclosure of which is incorporated, in its entirety, by this reference.

### BACKGROUND OF THE INVENTION

Osteoarthritis has historically been thought to be a traumatic or degenerative condition. The underlying causes of osteoarthritis being age, joint trauma, altered biomechanics, and obesity. Felson D. (2006) N Engl J Med 354: 841-848. Inflammation is now being recognized as a major factor in osteoarthritis. Sokolove J, et al., Ther Adv Musculoskelet Dis. 2013 April; 5(2):77-94. The anabolic/catabolic balance is distorted in arthritis with the destructive catabolic factors increased in amounts relative to the anabolic factors. Cytokines such as IL-1 and TNF-alpha produced by activated synoviocytes, mononuclear cells or by articular cartilage itself significantly up-regulate metalloproteinases (MMP) gene expression. Certain cytokines also blunt chondrocyte compensatory synthesis pathways required to restore the integrity of the degraded extracellular matrix (ECM). The catabolic cytokines IL-1 and TNF-alpha significantly up-regulate MMP-3. Other cytokines have beneficial effect on articular cartilage or inflammation. IL-4 largely prevents MMP-mediated aggrecan breakdown. van Lent P L et al. Osteoarthritis Cartilage. 2002 March; 10(3):234-43. IL-10 is an anti-inflammatory cytokine. Pestka S, et al., Annu. Rev. Immunol. 22:929-79.

The cartilage itself plays a role in the anabolic/catabolic homeostasis. The superficial zone of OA cartilage specimens, which is characterized by fibrillations, chondrocyte clusters, and degenerative matrix changes, contains a variable proportion of cells that immunostain for IL-1beta, TNF-alpha, and 6 different MMPs. These observations support the concept that cytokineMMP associations reflect a modified chondrocyte phenotype and an intrinsic process of cartilage degradation in OA. Tetlow L C, et al., Arthritis Rheum. 200, March; 44(3):585-94.

Therefore there are two major factors to consider in maintaining or restoring the health to synovial joints and articular cartilage; inflammation and the anabolic/catabolic balance. It is stated that the treatment of osteoarthritis is palliative with attention given to controlling the patient's pain. Treatment recommendations include weight loss, moderate exercise, and physical therapy. A number of oral medications have been advanced for the treatment of osteoarthritis. Among them are aspirin and acetaminophen to help relieve pain. Nonsteroidal anti-inflammatory drugs are intended to reduce pain and swelling. Intra articular injections of steroids and or hyaluronic acid shots have been advocated. In rare cases opioids have been used to reduce severe pain. In addition, there is no present disease modifying drugs for osteoarthritis as proposed for rheumatoid arthritis. Singh J., et al. (2012) Arthritis Care Res (Hoboken) 64:625-639.

The definitive treatment for end stage osteoarthritis over the years has been surgical in nature; debridement procedures, joint fusion, and more recently total joint replacement, all of which are tacit evidence of failure of the availability of earlier effective non-surgical measures.

In view of recent evidence, one treatment objective is to minimize the inflammatory response that is responsible for the catabolic cytokine production by way of lymphocytes and macrophages that produce the catabolic cytokines. The other treatment objective is the restoration of the anabolic/catabolic balance by affecting the synovium genetic makeup, the resultant synovial fluid chemistry and the response of the articular cartilage.

Any form of treatment intended for synovial joint health would need to demonstrate decreased inflammation, plus enhancement of the anabolic factors with reduction in the concentration of the corresponding catabolic factors. The effective alteration in these factors will show a response of the articular cartilage as demonstrated by histochemical analysis and various cartilage integrity grading systems.

The underlying problem may be the involvement of oxidative stress. Yudoh K, et al., Arthritis Res Ther. 2005; 7(2):R380-91. Therefore anti-oxidant reagents should be considered for the treatment of arthritis. Anthocyanins and anthocyanidins, including their primary metabolites are such reagents. Goodwin W, et al., J Orthop Res. 2010 August; 28(8):1057-63; Jensen G S, et al., J Medicinal Food 17 (11) 2014, 1-10.

### Factors in the Anabolic/Catabolic Balance:

Synovium: The normal synovial joint maintains a homeostatic balance of the anabolic/catabolic factors. These factors are present in the synovium, the synovial fluid and the articular cartilage. The synovium lines the joint cavity. It is a thin layer of very vascular fibrous adipose tissue. In health there is a thin layer covering of synovial cells. In disease the synovial cell layer proliferates to a multi-cell layer, even to the extent of forming villous projections. The synovium is the productive source of the synovial fluid composition. The synovium has the genetic potential to produce various growth hormones and cytokines.

Only recently has serious attention been given to the role of the synovium and the resultant chemical dynamics of osteoarthritis. Haraoui B., et al., Arthritis Rheum 34: 153-163; Gobezie R., et al. (2007) Arthritis Res Ther 9:R36.

The role of the synovium in osteoarthritis is of clinical and prognostic importance. Krasnokutsky S., et al. (2011) Arthritis Rheum 63: 2983-2991. Roemer F., et al. Ann Rheum Dis 70:1804-1809. The synovium in osteoarthritis plays a role in the production of catabolic cytokines. One source is the synovial macrophages. Bondeson J., et al., (2006) Arthritis Res Ther 8: R187. Fibroblast-like synoviocytes act as intermediate mediators of local inflammation. Studies have demonstrated that OA FLS produce inflammatory cytokines such as TNFα, IL-1β and MMPs. IL-1β and TNFα have been shown to induce cartilage degradation. Steenvoorden M., et al. (2007) Clin Exp Rheumatol 25: 239-245.

In osteoarthritis, both inflammatory and destructive responses are dependent largely on synovial macrophages that are cytokine-driven through a combination of IL-1 and TNF-alpha. Bondeson J. et al., Arthritis Res Ther. 2006; 8(6):RI87; Goldring S R, Goldring M B., Clin Orthop Relat Res. 2004 October; (427 Suppl):527-36.

### Synovial Fluid:

The synovial fluid is in part an ultra-filtrate of the blood plasma. As such it has the same molecules as the blood plasma. In the normal state, the synovial glucose and white blood cell counts would be lesser amounts than that of the blood. There should be no red blood cells in normal synovial fluid. The synovium and articular cartilage also contribute to the fluid's anabolic/catabolic chemical content.

### Articular Cartilage:

The articular cartilage in the normal state is avascular. It gains its nutrition by diffusion from the subchondral bone beneath and the bathing of the synovial fluid on the surface. It also has a genetic potential for production of various cytokines as well as pleuripotential stem cells.

### Growth Factors:

In synovial joints, numerous growth factors work in concert to regulate development and homeostasis of articular cartilage throughout life.

TGF-b1 stimulates synthesis of ECM and decreases catabolic activity of IL-1 and MMPs in cartilage. It also causes synovial proliferation and fibrosis. TGF-b1 induces chemotaxis of inflammatory leukocytes to synovium and is responsible for induction of osteophyte formation. TGF-b1 increases proliferation and ECM production and down regulates collagen type 1 gene expression. TGF-b has been shown to stimulate chondrocyte synthesis of collagens and proteoglycans and reduce the activity of IL-1 and opposes the inhibitory and catabolic effects of IL-1. Pujol J P, et al., Biorheology. 2000; 37(1-2): 177-84.

IGF-I stimulates extracellular matrix (ECM) synthesis, decreases matrix catabolism except in aged and OA cartilage, protective effect on synovium resulting in decreased thickening and decreased evidence of chronic inflammation and stimulates stem cell proliferation, increases expression of ECM and has an additive effect when combined with TGF-b. IGF-1 has shown promise as an anabolic reagent for cartilage healing; its chondroreparative nature. To date the reports have been in vitro studies or those in laboratory animals. IGF-1 has been shown to have an anti-apoptotic effect on cartilage cells. D'Lima D D, et al., J Bone Joint Surg Am. 2001; 83-A Suppl2(Pt 1):25-6. IGF-1 has been shown to inhibit chondrocyte apoptosis induced by collagenase treatment. Lo M Y et al., J Orthop Res. 2004 January; 22(1): 140-4.

PDGF: (Platelet Derived Growth Factor) has no adverse effect in normal joint cartilage and has no adverse effect in normal joint synovium. It induces stem cell proliferation. Fortier L A, et al., Clin Orthop Relat Res (2011) 469:2706-2715; Goodrich L R et al., J Bone Joint Surg Br. 2007 May; 89(5):672-85; Schmidt M B, et al., Osteoarthritis Cartilage. 2006 May; 14(5):403-12; Tyler J A, Biochem. J. (1989) 260 (543-548).

A model system of explanted cartilage has been used in vitro to determine whether insulin-like grown factor 1 (IGF 1), which promotes matrix formation is effective in the presence of cytokines such as interleukin 1 (IL1) and tumor necrosis factor (TNF), which induce net matrix depletion. IGF 1 induced a dose-dependent 2.5-fold stimulation of proteoglycan synthesis, with a half-maximal dose of 25 ng/ml. A similar relative increase occurred in response to IGF 1 (10-100 ng/ml) in cartilage cultured also with IL1 or TNF (5-500 pM). There was no detectable qualitative change in the average molecular size or charge of the aggregating proteoglycan synthesized by explants exposed to IGF 1 alone or with IL1 or TNF. The increased production of prostaglandin E2, which is initiated when IL1 or TNF bind to the chondrocytes, was the same in the presence or absence of IGF 1. The time taken for 50% of pre-labelled proteoglycan to be released from the explants (t½) increased in the presence of IGF 1 (100 ng/ml) from 21 to 32 days in control cultures and from 8 to 26 days in cartilage cultured with IL1 (50 pM). It is concluded that IGF 1 enhances the synthesis of aggregating proteoglycan in cartilage exposed to cytokines and can directly decrease both the basal and the cytokine-stimulated degradation of proteoglycan in cartilage.

TGF-beta, IGF-1 and FGF-2, in particular, have been characterized as potential chondroprotective agents. Malemud C J. Drugs Aging. 2010 Feb. 1; 27(2):95-115. It may be that there is synergistic effect of the various growth hormones.

In the normal joint all these factors are in balance. Therefore any attempt to enhance or restore synovial joint health must address these two factors; inflammation and the anabolic/catabolic balance. Based upon the medical literature the following issues need to be addressed. To date, no agent has been approved for the prevention or treatment of OA disease progression. Synovitis occurs very early in OA. Synovial hyperplasia can be three layers thick and as such is a poor prognostic factor. High levels of catabolic cytokines are the result of the synovitis: TNF, IL-1b, MMP-1, MMP-3, and IL-6. Synovial macrophages that produce the cytokines need to be reduced. Bondeson, J. et al., Arthritis Res Ther. 2006; 8(6):R187. PGE2 is involved in inflammation. Martel-Pelletier J., et al., (2003) Semin Arthritis Rheum 33: 155-167. No studies to date have targeted the very early OA at a time when anti-inflammatory intervention might be most effective. Steenvoorden M., et al. (2007) Clin Exp Rheumatol 25: 239-245.

There thus remains a need for a disease-modifying OA drug (DMOAD), a reagent that addresses the synovium and the effects in the synovium in the synovial fluid. The drug needs to alter synovial fluid anabolic/catabolic balance of the cytokines in the osteoarthritic joint. Ideally the drug would have an anabolic effect on cartilage health, nutrition, and protection. The present invention meets these needs.

Background on Anthocyanins/Anthocyanidins and their Metabolites:

Anthocyanidins and anthocyanins are phytochemicals. Previous in vitro testing was performed with bovine cells and 100 pM concentrations of cyanidin CI, delphinidin-3-0-glucoside (D-3-G), and cyanidin-3-glucoside (C-3-G) in assessing the 35S incorporation over time and various conditions. The results showed no incorporation at that dose level in 6 hours with or without presence of IL-1 a. At 12 and 24 hours only D-3-G reversed the IL-1 a suppression effect on the 35S uptake. At 24 hours the three anthocyanins tended to suppress the 35S uptake. However when combined with TGF-b, only C-3-G showed a synergistic effect on 35S uptake. It is possible that 6, 12, and 24 hours were too soon at this dose to have an effect on bovine cells. It is also possible the parent anthocyanin molecule may have remained unchanged and not converted to the potentially more active metabolites. The results showed that different anthocyanidins reactive differently under various conditions. D-3-G reversed the IL-1 a effect and only C3G had synergistic effect with TGFβ. D'Lima D., Anabolic and anti-arthritic effect of anthocyanins, Final Report. Jan. 19, 2010, Shiley Center for Orthopedic Research and Education at Scripps Clinic, La Jolla, Calif.

Research supporting U.S. Pat. No. 8,263,069 demonstrated that a certain phytochemicals and or their metabolite cause the human synovial explants in tissue culture to turn the gene on for IGF-1 and increase this hormone in the culture media.

It is important to recognize that anthocyanins and anthocyanidins are very unstable in nature. For instance cyanidin-3-glucoside when ingested by a human, 73% of the parent 15 anthocyanin becomes the metabolite protocatechuic acid in the blood stream in 30 minutes. Vitaglione P. et al., J Nutr. 2007 September; 137(9):2043-8.

Cyanidin-3-Glucoside is unstable in tissue culture maintaining integrity for 24 hours. The same material in a proprietary commercial form, ProC3G™ (Chromadex, Inc. Irvine, Calif.) will remain on an experimental rodent skin wound scab for 48 hours. Cyanidin chloride is very unstable in matter of a few minutes when exposed to an environment with a basic pH. The same is so with protocatechuic acid and 2,4,6 trihydroxybenzaldehyde, that are all a part of the anthocyanin, anthocyanidin metabolite degradation chain. Therefore the chemical effect in a biological environment for any anthocyanin or anthocyanidin may be due to some proportion of their main metabolites. The action may be due to a combination of the parent anthocyanidin and he metabolites, including Cyanidin-3-Glucoside, PCA and possibly vanillic and hippuric acid and other metabolites.

Cyanidin-3-Glucoside is rapidly absorbed and metabolized in adult men. The oral intake of cyanidin-3-glucoside in adult men found the parent molecule at a 32.0% concentration at 0-7 hours. The remainder was metabolites of the parent molecule. The same percentages were found in the urine at 24 hours. Kay C D, et al., J Nutr. 2005 November; 135(11):2582-8.

Protocatechuic acid is a more stable molecule. However, it is cleared fairly rapidly from blood plasma with an initial half-life of 3 minutes and terminal half-life of 16 min in the human. Ronald Prior, PhD. Personal communication.

The inventor has discovered that the oral ingestion of certain anthocyanins/anthocyanidins and/or their metabolites will decrease inflammation in an osteoarthritic joint and alters the anabolic/catabolic state of the joint, while providing chondro-nutrition and chondro-protection to the articular cartilage. This treatment method may have the potential to be a Disease Modifying Osteoarthritic Drug (DMOAD).

### SUMMARY OF THE INVENTION

The present invention provides oral formulations useful for promoting healing and reducing inflammation of an injured or arthritic joint. The formulations can be taken as a nutraceutical, as a prophylactic or as a therapeutic treatment. A prophylactic course can start before surgery of a joint occurs and continue on for a time period after the surgery. A therapeutic course can start after an injury occurs or after surgery or after inflammation has begun. The formulations are as described herein below in more detail. In certain embodiments, the active ingredient comprises, consists essentially of or consists of an anthocyanin or anthocyanidin, or their metabolites or mixtures thereof. An exemplary active ingredient consists of C3G or PCA.

The present invention provides methods of treating or decreasing inflammation of a joint in a mammalian subject, by orally administering to the subject a composition comprising a formulation or composition of the present invention. The composition has the effect of decreasing levels of MMP1, TFG-beta, and MMP13 in the synovial fluid of the joint and increasing the levels of TIMP-1, VEGF, IL-10 and Il-4 in the synovial fluid of the joint to cause a reduction of inflammation in the joint.

The inflammation in the joint is due to an injury or trauma to the joint or due to arthritis of the joint or due to inflammatory arthritis.

In certain embodiments, the oral administration comprises administering an oral daily dose for at least 6 weeks of a dose of 0.062 millimoles C3G per kg body weight or (30 mg/kg). In certain embodiments, the oral administration comprises administering a daily dose for at least 6 weeks of a dose of 0.177 mmoles PCA per kg body weight (26.4 mg/kg).

In certain embodiments, the oral administration comprises administering a daily dose for at least 10 weeks of a dose of 0.062 millimoles C3G per kg body weight (30 mg/kg). In certain embodiments, the oral administration comprises administering a daily dose for at least 10 weeks of a dose of 0.177 mmoles PCA per kg body weight (26.4 mg/kg).

In certain embodiments, the oral administration comprises administering a daily dose for at least 4 weeks of a dose of 0.062 millimoles C3G per kg body weight (30 mg/kg) starting after the injury. In certain embodiments, the oral administration comprises administering a daily dose for at least 4 weeks of a dose 0.177 mmoles PCA per kg body weight (26.4 mg/kg) starting after the injury. The treatment can begin any time after injury or inflammation occurs. For example the daily dose can begin weeks (even 3 weeks) after the injury or surgery or after the inflammation has begun.

In certain embodiments, the oral administration comprises administering a daily dose for some time period prior to joint surgery of a dose of 0.062 millimoles C3G per kg body weight (30 mg/kg), or 0.177 mmoles PCA per kg body weight (26.4 mg/kg) and continuing on for some time after the surgery. As one illustrative example, the daily dose may begin at least one week before surgery and continue on for at least 4 weeks after surgery at the same dose.

In certain embodiments the oral daily dosage ranges from 0.035 to 0.100 millimoles C3G per kg of body weight. In certain embodiments the oral daily dosage ranges from 0.035 to 0.100 millimoles an anthocyanin or an anthocyanidin or combination thereof per kg of body weight. The oral daily dose can be given before surgery or before injury or before inflammation as occurred as a prophylactic treatment. The oral daily dose can be given during surgery and can be continued on after surgery or injury until the inflammation has been reduced satisfactorily and until the injury has healed. The oral daily dose can be given on a long term basis in cases of chronic inflammation due to arthritis or other causes of inflammation.

In certain embodiments the oral daily dosage ranges from 0.100 to 0.200 mmoles PCA per kg body weight. In certain embodiments the oral daily dosage ranges from 0.100 to 0.200 mmoles of an anthocyanin metabolite or anthocyanidin metabolite or combination of metabolites 30 per kg body weight. The oral daily dose can be given before surgery or before injury or before inflammation as occurred as a prophylactic treatment. The oral daily dose can be given during surgery and can be continued on after surgery or injury until the inflammation has been reduced satisfactorily and until the injury has healed. The oral daily dose can be given on a long term basis in cases of chronic inflammation due to arthritis or other causes of inflammation.

The present invention also provides a method of improving the catabolic/anabolic state of an inflamed joint in a mammalian subject, by orally administering to the subject a composition of the present invention. The improved catabolic/anabolic state is brought about by a process selected from the group consisting of reduction of levels of MMP3, MMP-1, MMP13, IL-1B, TGF-beta, TNF-alpha, ADAMTS-5, in synovial fluid or synovium; and/or an increase of levels of IGF-1, lubricin, TIMP-1, VEGF, Il-10, IL-4, collagen II and/or aggrecan in synovial fluid or synovium of the inflamed joint.

The present invention also provides a method of decreasing the numbers of macrophages, lymphocytes and polymorphonuclear leukocytes in synovial fluid of an inflamed joint by orally administering to a subject a composition as described herein.

The invention also provides a method treating an injured or arthritic joint by promoting healing of the cartilage of the joint in a mammalian subject, by orally administering to the subject a composition described herein, wherein the composition has the effect of increasing in the cartilage of the injured or arthritic joint expression levels of lubricin, aggrecan and collagen II to promote healing and to treat the injured or arthritic joint.

The invention also provides a method of decreasing levels of C reactive protein (CRP) in the plasma in a subject having an inflamed or injured joint, by orally administering to the subject a composition as described herein wherein the administration has the effect of decreasing levels of CRP in the plasma of the subject.

Also described herein is a method of providing a prophylactic treatment to a subject to reduce inflammation, wherein a composition of the invention is provided to the subject in advance of a surgery to the joint. The treatment may continue during the surgery as well as after the surgery to continue to reduce inflammation.

Further described herein is a method of providing a therapeutic treatment to a subject to reduce inflammation after surgery, injury or after inflammation has begun, wherein a composition of the invention is provided to the subject after surgery or injury to the joint, or after inflammation has already occurred.

### BRIEF DESCRIPTION OF THE FIGURES

Figure legends:

Control: test subject did not receive any medication.
CI 1-42: prophylactic C3G group (test subjects received 30 mg C3G/kg Body weight (“BW”) Chromadex Pro3CG™ by mouth 7 times per week for 42 days).
PCA 1-42: prophylactic PCA group (test subjects received 26.4 mg (PCA)/kg BW by mouth 7 times per week for 42 days).
CI42-70: Therapeutic C3G group (test subjects received 30 mg (C3G)/kg of body weight Chromadex™ Pro3CG™ by mouth (starting on day 42 after surgery through day 70) 7 times per week for 4 weeks.
PCA 42-70: Therapeutic PCA group (test subjects received 26.4 mg (PCA)/kg bodyweight by mouth (starting on day 42 after surgery through day 70) 7 times per week for 4 weeks.

FIG. 1 (synovium) shows that MMP-13 levels were slightly decreased in the C3G therapeutic group and very slightly decreased in the PCA prophylactic group. The levels seemed to be increased slightly in the PCA therapeutic and the C3G prophylactic group. However, the levels of change appear not to be statistically significant.

FIG. 2A shows that levels of IL-1 beta were decreased in the synovial fluid in all groups.

FIG. 2B shows that TGF-beta was decreased in the synovial fluid in all groups.

FIG. 2C shows that MMP3 was decreased in the synovial fluid for therapeutic groups for C3G and PCA.

FIG. 3 shows that MMP-3 expression in cartilage was reduced in the both the therapeutic and the prophylactic G3G and PCA groups.

FIG. 4 provides results of histochemical staining for the presence of MMP-3 and expression in cartilage. The negative control shows no staining. Group 1, is a positive control. Group 2 (C3G prophylactic) shows very minimal positive stain for MMP3 with a single arrow pointing to the positive stain. Groups 3 (PCA prophylactic) and Group 4 (C3G therapeutic) and Group 5 (PCA therapeutic) show no staining.

FIG. 5A shows that TNF-alpha levels showed a slight increase in the synovial fluid for the C3G and PCA therapeutic groups and a slight decrease in both of the therapeutic groups. However the differences seen are not statistically significant. However, notably the reduction occurred due to the longer treatment time; 6 weeks in the prophylactic group as opposed to 4 weeks in the therapeutic groups. Thus, a viable therapy would preferably have a minimum 6 week duration required for depression of TNF-α.

FIG. 5B shows that IFG-1 levels in the synovial fluid was not significantly changed in all groups. However, notably the levels were slightly elevated in the 6 week prophylactic group, but not the 4 week therapeutic groups. This may hint that the timing of the treatment may be important. Further the results shown in this graph also has to be balanced by the synovial fluid IGF-1 receptor amounts detected by ELISA. IGF-1 was active in preserving the patellar cartilage.

FIG. 6A shows that ADAMTS-5 was decreased in the synovial fluid in both C3G groups and in the therapeutic PCA group.

FIG. 6B shows that MMP-13 was decreased in the synovial fluid for all groups.

FIG. 6C shows that the TIMP-1 levels were elevated in both C3G groups and lowered in the PCA therapeutic group. However, statistical significance was not evident.

FIG. 7 shows that MMP-I levels in the synovial fluid were decreased significantly in all groups.

FIG. 8A shows that TGF-beta was increased in the synovium only in the C3G therapeutic group.

FIG. 8B shows that IGF-1 levels in the synovium was increased in all groups.

FIG. 8C shows that lubricin was increased in 3 of the 4 groups in the synovium (increase in both C3G groups and increase in the prophylactic PCA group).

FIG. 9 A shows that levels of IL-1 beta were decreased in the synovium of the C3G and PCA therapeutic groups, but were increased in the two prophylactic groups.

FIG. 9B shows that MMP-3 levels were reduced the synovium in all four groups.

FIG. 9C shows that IL-6 was decreased in the synovium in all groups.

FIG. 9D shows that TNF-alpha levels were decreased in the synovium for all groups.

FIG. 10 shows that TIMP-1 gene expression was increased in the synovium in all four test groups.

FIG. 11 shows that the therapeutic C3G group showed a decrease in MMP-1 in the synovium.

FIG. 12 shows that ADAMTS-5 gene expression was decreased in the synovium in the C3G and the PCA therapeutic groups.

FIG. 13A shows that MMP-3 gene expression was decreased in the synovium as detected by real time PCT in all groups.

FIG. 13B shows that IGF-1 gene expression as detected by real-time PCR was increased in all groups.

FIG. 14 shows that expression of IL-Beta in cartilage is decreased in all four groups.

FIG. 15 shows the results of immunohistochemical staining for IL-Beta in rabbit tissue supporting FIG. 14.

FIG. 16 shows that collagen II expression in cartilage is increased in all4 groups.

FIG. 17 provides the underlying immunohistochemical staining supporting FIG. 16.

FIG. 18 provides the underlying immunohistochemical staining supporting FIG. 19.

FIG. 19 shows that lubricin expression was increased in the cartilage of all four groups.

FIG. 20 shows increased expression of aggrecan in patellar cartilage of all four groups.

FIG. 21 provides a graph of ICRS histological visual scale scores for the surface of the cartilage. Scores for all four groups improved.

FIG. 22 provides a graph of ICRS histological visual scale scores for the cell matrix. Scores for all four groups improved.

FIG. 23 provides a graph of ICRS histological visual scale scores for cell distribution. Cell distribution for all four groups improved.

FIG. 24 provides a graph of ICRS histological visual scale scores for the cell viability. Cell viability remained the same for all four groups.

FIG. 25 provides a graph of ICRS histological visual scale scores for the subchondral bone. Scores for all four groups improved.

FIG. 26 provides a graph of ICRS histological visual scale scores for the Mineralization. Scores for all four groups improved.

FIG. 27 shows that the levels of VEGF in the synovium were increased in all four groups.

FIG. 28 shows that the levels of IL-10 in the synovial fluid were increased in all four groups.

FIG. 29 shows that the levels of IL-4 in the synovial fluid was increased in all four groups.

FIG. 30 shows that the total white blood cell count in the synovial fluid was decreased in all 4 study groups.

FIG. 31A shows that the number of PMN s was also reduced in the synovial fluid of all four groups, although most noticeable in the C3G groups.

FIG. 31B shows that the number of macrophages was also decreased in all groups.

FIG. 31C shows that the number of lymphocytes in the synovial fluid was also decreased in 3 of the groups (both the G3G and PCT therapeutic group and the PCA prophylactic group).

FIG. 31D shows that the total white blood cell count in the synovial fluid was decreased in all 4 study groups.

FIGS. 32A-C show Phenolic acid excretion in rabbits following oral gavage of control, cyanidin-3-glucoside (C-3-G)(30 mg/kg BW or Protocatechuic acid (3,4-dihydroxybenzoic acid)(26.4 mg/kg) expressed as micrograms per mg creatinine in the urine sample. FIG. 32A is for 3,4-HBA excretion. FIG. 32B is for Vanillic acid and 32C is for Hippuric acid.

### DETAILED DESCRIPTION OF THE INVENTION

The inventor has shown that oral administration of an anthocyanin or anthocyanidin, or their metabolites specifically cyandin-3glyoside (C3G) and protocatechuic acid (PCA) has positive effects, including anti-inflammatory, chondro-nutritive, chondro-protective and has the ability to favorably alter the catabolic/anabolic balance in an injured/arthritic joint.

An arthritic joint was created in test mammals (rabbit) and then the animals were given oral doses of C3G or PCA (ether a prophylactic regimen or a therapeutic regimen—as will later be described) and then various tests were performed on the cartilage, the synovial fluid and the synovial tissue (synovium). The studies ran showed a favorable/positive change in WBC counts, in the anabolic/catabolic chemistry and cartilage preservation in the face of severe degenerative arthritis. A study of the C-reactive protein (CRP) levels in the plasma showed that the prophylaxis treatment regimen (daily for 6 weeks following injury) lowered CRP levels to that of a normal range, thus indicating a positive anti-inflammatory effect.

These tests showed that the oral administration was able to favorably alter the genetic make up the synovium as evidence by seeing an increase in IGF-1, lubricin, TIMP-1, collagen II and aggrecan; and seeing a decrease in MMP-3, MMP1, IL-6, TNF-alpha, and ADAMTS-5. The articular cartilage was studied and gene expression was measured as well histochemical studies were performed. It was shown that the oral administration provided a chondro-nutritive and chondro-protective benefit (e.g. increased expression in the cartilage of lubricin, aggrecan, and collagen II). Studies also showed that in the synovial fluid, the oral administration had positive anti-inflammatory effects (e.g. increased IL-10 and IL-4; and decreased white blood cell counts including PMNs, macrophages, and lymphocytes) and provided favorable alterations in anabolic/catabolic chemistry (e.g. increase in VEGF and TIMP-1 and reduction in MMP-1, MMP3, IL-1B, TGF-B, TNF-alpha, MMP-13, and ADAMTS-5).

Anthocyanins include cyanidin-3-glucosidase or delphinidin-3-glucosidase, cyanidin-3-galactosidase, and pelargonidin-3-galactosidase. Anthocyanidins include cyanidin, delphinidin, pelargonidin, malvidin and petunidin. Metabolites of anthocyanidin include, but are not limited to protocatechuic acid (PCA) and, 2,3,4 trihydroxybenzaldehyde.

The term “chondroprotective” or “chondroprotective agent” as used herein refers to a process, substance or molecule that inhibits or reduces the degradation of cartilage or chondrocytes. The compositions of the invention are chondroprotective in that they reduce various inflammatory cytokines to reduce inflammation and thereby reduce the degradation of cartilage or chondrocytes. The compositions also increased the all important surface lubricant, lubricin, which promotes joint surface gliding, thereby reducing peak axial loads on the articular cartilage.

The term “chondronutritive” or “chondronutritive agent” as used herein refers to a process, substance or molecule that activates a cartilage cell to produce or enhances the production of glucopolysaccharides. The compositions of the invention are chondronutritive in that they increase the production of collagen I, aggrecan and lubricin in the synovium.

The term “chondroreparative” or “chondroreparative agent” as used herein refers to a process, substance or molecule that causes cartilage to repair, such as with fibrocartilage. The compositions of the invention are chondroreparative because they increased the IGF-1 in the synovium and synovial fluid. IGF-1 arrests programmed cell death, apoptosis. In addition the compositions by way of optimizing the anabolic/catabolic nature of the synovial joint environment, they thereby maximize the chondronutrition and condroprotection for cartilage healing by the increase in IGF-1 presence and, in the case of surgery (open or arthroscopic) or arthrocentesis the presence of bleeding providing blood, a common denominator for wound healing. See Johnson L L. Characteristics of the immediate post arthroscopic blood formation in the knee joint. Arthroscopy. 1991; 7(1):14-23.

The term “chondrorestorative agent” as used herein refers to a process, substance or molecule that causes cartilage to be restored to its normal hyaline pattern or nature. A chondrorestorative agent restores or improves normal activities or functions to the cartilage. The compositions of the invention provide the optimal environment for chondrorestoration. There is evidence of increased biomarkers (lubricin, aggrecan and collagen II) in the articular cartilage of the most severely surgically induced degenerative joint. These three provide the necessities and ingredients for the promotion of healing and restoration. Restoration of cartilage typically progresses through a fibrous reparative state to one of fibrocartilagenous nature. After time, even years, the reparative nature transforms to hyaline cartilage. Johnson L L, Delano M C, Spector M, Jeng L, Pittsley A, Gottschalk A. The biological response following autogenous bone grafting for large volume defects of the Knee: index surgery through 12-21 year follow-up. Cartilage, Volume 3 Issue 1. January 2012. Pp 85-98. First published on Aug. 16, 2011 as DOI: 10.1177/1947603511413568.

The term “pharmaceutically acceptable carrier” as used herein refers to the acceptance or use of the carrier in the pharmaceutical industry. Preferably the carrier is approved by the Federal Drug Administration (FDA) for use in humans. Exemplary carriers include physiological solutions including but not limited to glucose, dextrose, normal saline, phosphate buffered saline (PBS) or Ringer's solution.

The term “therapeutically effective amount” as used herein refers to an amount of an active ingredient that produces the intended result.

C3G includes pure C3G and a commercially available formula offered by Chromadex™ (called PROC3G), which has 35% C3G along with a small amount of other inert botanicals and small amount of phytochemicals as indicated in Chromadex™ documents. The oral dose can include, but is not limited to a tablet, capsule, powder, powder reconstituted in a liquid suitable for ingestion. Pharmaceutically acceptable carriers, preservatives, emulsifiers, stabilizers and other additives may be added.

The present invention provides a formulation useful in a method of decreasing inflammation of a joint in a mammalian subject. The present invention also provides methods of decreasing inflammation of a joint in a mammalian subject. The inflammation in the joint can be from an injury or trauma (including post-surgery inflammation) or from arthritis. Thus the joint can be injured or can be an arthritic joint.

Methods involve orally administering to the subject a composition comprising an active ingredient selected from the group consisting of ProC3G™, cyanidin-3-glucoside (C3G) or protocatechuic acid (PCA) or a combination thereof. In certain embodiments, the active ingredient consists of C3G. In other embodiments the active ingredient consists of ProC3G™. In other embodiments, the active ingredient consists of PCA. In yet other embodiments, the active ingredient consists of a mixture or C3G and PCA or consists of a mixture of ProC3G™ and PCA. In certain embodiments the active ingredient is the parent anthocyanin, anthyocyanidin or their metabolites, or a combination thereof. For example, the active ingredient may be a combination of more than one anthocyanins; a combination of more than one anthocyanidin; a combination of anthocyanins and anthocyanidins; a combination of at least one anthocyanin and at least one of its metabolites; a combination of at least one anthocyanidin and a least one of its metabolites; and a combination of at least one metabolites of at least one anthocyanin and at least one metabolite of at least one anthocyanidin. The active ingredient may consist of one anthocyanin or one anthocyanidin or one metabolite. For example, the active ingredient may consist of C3G or PCA.

In certain embodiments the active ingredient is a mixture of at least one anthocyanin and at least one metabolite of an anthocyanidin. In certain embodiments the active ingredient is a mixture of at least one anthocyanidin and at least one metabolite of an anthocyanin.

In some embodiments the active ingredient is a mixture of C3G and PCA.

In one embodiment, the PCA is present at about 97% of the total active ingredient.

In other embodiments the active ingredients are metabolites of anthocyanins or mixtures of metabolites. Metabolites include, but are not limited to PCA, 3, 4 hydroxybenzoic acid, 3-Hydroxybenzoic acid, 4-Hydroxybenzoic acid, Benzoic acid-4-glucuronide, PCA-3-glucuronide, PGA-4-glucuronide, PCA-3-sulfate, PCA-4-sulfate, Vanillic acid (VA), IsoVA, isoVA-3-glucuronide, VA-4-glucuronide, IsoVA-3-sulfate, VA-4-sulfate, 4-Hydroxyphenylacetic acid, 3,4-Dihydroxyphenylacetic acid, 4-Hydroxybenzaldehyde, 3,4-Dihydroxybenzaldehyde, Caffeic acid, Hippuric acid, and Ferulic acid.

Most prior studies showing health benefit were mixtures, compounds and extracts of many phytochemicals each of low concentration. There presently are no 100% concentrations of the parent anthocyanins nor the metabolite, commercially or otherwise. There is a 95% (+/−) C3G available, but it is very expensive. Since the percentage of anthocyanin is low in berries (7 mg/100 grams) it is not likely or practical for anyone to ingest a large enough volume of berries to get the same benefit as the concentrations used in the methods of the present invention.

In the present invention, in certain embodiments the dose is 25-30 mg/kilogram of bodyweight. In certain embodiments the duration of the treatment is for 6 weeks and the dose is given 7 days per week. In certain embodiments, the duration of treatment is for 4 weeks and the dose is given 7 days per week. In certain embodiments, the treatment regimen starts immediately after injury and proceeds for at least 6 weeks. In certain embodiments the treatment regimen starts 6 weeks after injury and proceeds for at least 4 weeks. In certain embodiments, the treatment regimen starts immediately after injury and proceeds for at least 10 weeks.

In certain embodiments, the route of administration is oral. Powdered active ingredient can be mixed with a suitable liquid for drinking or gavage or alternatively, the active ingredient can be in the form of a pill or capsule. The active ingredient may also be mixed with other solid eatable ingredients, such as for an example, in a nutrition/snack bar.

In certain embodiments, the treatment is provided before surgery as a prophylactic treatment or prophylactic nutraceutical. In certain prophylactic therapies, the treatment is continued to be provided for at least 6 weeks after the surgery. In certain embodiments, the treatment is every day.

In certain embodiments, the treatment is provided after surgery. In certain embodiments the treatment is a peri-surgical treatment (during surgery) or in conjunction with surgery.

In certain embodiments the oral formulation/treatment is given to a subject as an over the counter oral nutraceutical where the subject has existing arthritis or an existing joint injury who is suffering from inflammation in the joint.

It was previously thought that anthocyanins has little bioactivity due to the less than 1% recovery of the dose. (Kay C D, et al. (2004), Br J Nutr 91: 933-942; Manach C, et al. (2005), Am J Clin Nutr 81: 230S-242S; McGhie T K, Walton M C (2007), Mol Nutr Food Res 51: 702-713). A subsequent study showed that protocatechuic acid was the main metabolite of anthocyanins. Carbon labeling showed that Cyanidin-3-glucoside was in 30 minutes 74% protocatechuic acid in the plasma. (Vitaglione P, et al. (2007), J Nutr 137: 2043-2048). Furthermore, the bioavailability of C3G was established as 12.4%, based on the recovery of the 13C-label in the urine and breath. (Czank C, et al., The American journal of clinical nutrition 97 (5), 995-1003).

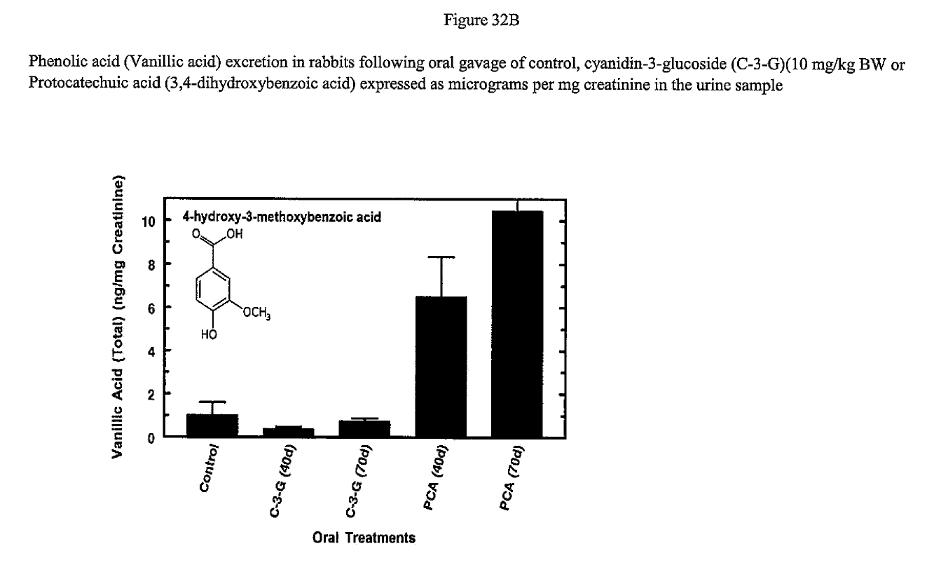

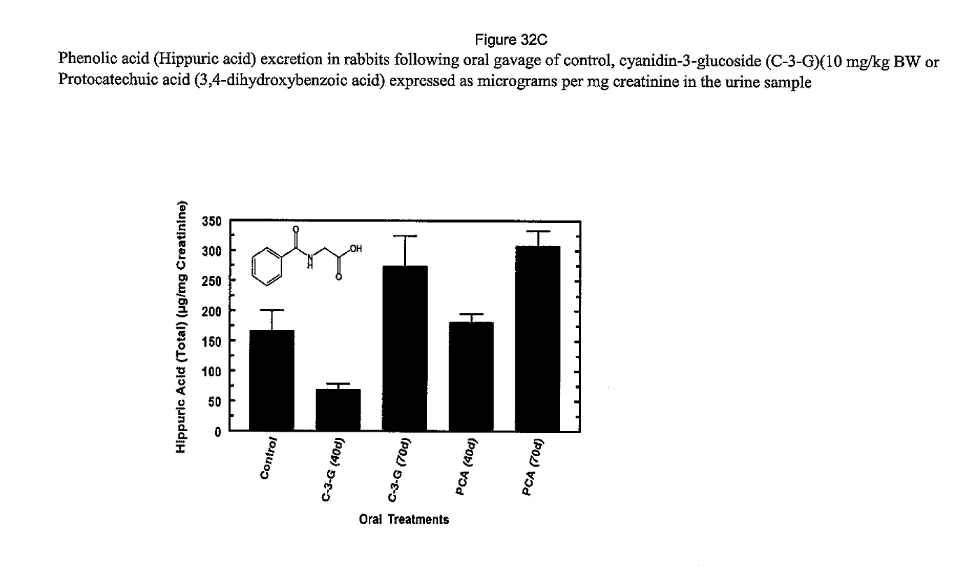

Recent publications have more clearly established the metabolites as the bioavailable reagents. (de Ferrars R M, et al., Br J Pharmacol. 2014 July; 171(13):3268-8; Kalt W, et al., J Agric Food Chem. 2014 Jan. 27; Pimpão R C, et al., Mol Nutr Food Res. 2014 July; 58(7):1414-25 (Epub 2014 Apr. 17)). These publications are from the nutraceutical literature. Following ingestion of anthocyanins, there is a continuous cascading of metabolism. The primary degradants were protocatechuic acid and phloroglucinaldehyde. Then there is phase II conjugates and on to the likes of ferulic acid, vanillic acid, hippuric acid. See FIG. 32 A-C. Hippuric acid is the most abundant metabolite.

In addition, it was reported that the duration of the metabolites far exceeded in duration in the body the parent anthocyanin. (de Ferrars R M, et al., Br J Pharmacol. 2014 July; 171(13):3268-82). The peak levels of the metabolites after fruit puree ingestion was 2 hours and lasted as long as 48 hours in the plasma. (de Ferrars R M, et al., Br J Pharmacol. 2014 July; 171(13):3268-8; Kalt W, et al., J Agric Food Chem. 2014 Jan. 27; Pimpão R C, et al. Mol Nutr Food Res. 2014 July; 58(7):1414-25 (Epub 2014 Apr. 17)).

The excretion route is both direct though the intestine to the feces, but also to the blood and exits via the urine, saliva and breath. (Czank C, et al., The American journal of clinical nutrition 97 (5), 995-1003; de Ferrars R M, et al., Br J Pharmacol. 2014 July; 171(13):3268-8; Kalt W, et al., J Agric Food Chem. 2014 Jan. 27; Pimpão R C, et al., Mol Nutr Food Res. 2014 July; 58(7):1414-25 (Epub 2014 Apr. 17)).

Therefore, any physiological or pharmacological effect is probably due to in part to the parent anthocyanin, but more likely the abundance of the continuous cascading of the metabolites. Accordingly, methods of the invention comprise oral administration of metabolites of anthocyanins/anthocyanidin (or mixtures of metabolites) as well as the anthocyanin/anthocyanidin (or mixtures thereof) itself. For example, a parent anthocyanin may be cyanidin-3-glucoside. Metabolites often called phase II conjugates, include protocatechuic acid (PCA) is one and phloroglucinaldehyde (PGA). Then there are many subsequent metabolites, and as many as 35 have been identified. Hippuric acid is one of these metabolites. R M de Ferrars reports in Br J Pharmacol. 2014 July; 171(13):3268 that “given the degradation, short t½ and low Cmax of the parent anthocyanins, the observed cardiovascular benefits of anthocyanin consumption are probably the consequence of the metabolites, which are present within the circulation for significantly longer and at higher concentrations than the parent anthocyanins.” De Farrar reports a total of 35 analytes (including the parent C3G) were identified in the serum, urine and fecal samples collected over 48 hours post-consumption. De Ferrars notes that his study “indicates that the majority of the label is present in the circulation as lower molecular weight 13C-labelled phenolic metabolites of the parent C3G.” And he further notes that “the parent anthocyanin only represented 2% of the total metabolites found in the circulation and was only present for a relatively short period of time (tVz, 0.4 h), thus suggesting that anthocyanin bioactivity is likely to be mediated by high concentrations of its phenolic intermediates as opposed to the parent structure. De Ferrars noted that hippuric acid was identified as the major metabolite of anthocyanins in his study. De Ferrars opines that his studies suggest that the bioactivity of anthocyanins is likely attributed to their phenolic metabolites.

The inventors have shown, as discussed below in the examples and the figures that the oral composition had the effect of decreasing levels of MMP1, TFG-beta, and MMP13 in the synovial fluid of the joint and increasing the levels of TIMP-1, VEGF, IL-10 and Il-4 in the synovial fluid of the joint to cause a reduction of inflammation in the joint.

The inventors also were able to demonstrate that the oral administration had the effect of improving the catabolic/anabolic state of an inflamed joint in a mammalian subject. This was evidenced by seeing a reduction of levels of MMP3, MMP3, MMP-13, IL-1B, TGF-beta, TNF-alpha, ADAMTS-5, the numbers of macrophages, lymphocytes and polymorphonuclear leukocytes in synovial fluid or synovium; and/or seeing an increase of levels of iGF-1, lubricin, TIMP-1, VEGF, 11-10, and/or IL-4 in synovial fluid or synovium.

The invention also provides a method of reducing MMP 3 levels in synovial fluid, synovium and cartilage of an inflamed joint by orally administering to a subject a composition comprising an active ingredient selected from the group consisting of ProC3G™, cyanidin-3-glucoside (C3G) or protocatechuic acid (PCA) or a combination thereof.

The invention also provides methods of increasing levels of IGF-1 in the synovium or increasing gene expression of IGF-1 in the synovium of an inflamed joint; methods of decreasing levels of MMP-1 in synovial fluid and synovium; methods of decreasing levels of II-1B in synovial fluid and decreasing gene expression of II-1B in the synovium of an inflamed joint; methods of decreasing levels of TGF-beta in synovial fluid of an inflamed joint; methods of increasing levels of lubricin in synovial fluid and synovium of an inflamed joint, and increasing gene expression of lubricin in the synovium of an inflamed joint; methods of decreasing levels of TNF-alpha in synovial fluid and synovium of an inflamed joint; methods of decreasing levels of ADAMTS-5 in synovial fluid and decreasing gene expression in synovium; method of decreasing levels of MMP-13 in synovial fluid of an inflamed joint; methods of increasing levels of TIMP-1 in synovial fluid or increasing gene expression in synovium; methods of increasing levels of VEGF in the synovium of an inflamed joint; methods of increasing levels of Il-10 in synovial fluid of an inflamed joint; method of increasing levels of IL-4 in synovial fluid of an inflamed joint; methods of decreasing the numbers of macrophages, lymphocytes and polymorphonuclear leukocytes in synovial fluid of an inflamed joint; methods of decreasing levels of IL-6 in the synovium of an inflamed joint; methods of decreasing MMP-3 in synovial fluid and/or synovium in an inflamed joint; methods of decreasing gene expression of MMP-3 in the synovium of an inflamed joint; methods of increasing collagen II expression in the synovium of an inflamed joint and methods of increasing aggrecan expression in the synovium of an inflamed joint.

The methods involve orally administering to a subject a composition of the present invention comprising an active ingredient selected from the group consisting of ProC3G™, cyanidin-3-glucoside (C3G) or protocatechuic acid (PCA) or a combination thereof.

Methods of the invention may involve other active ingredients as described below. In certain embodiments, the active ingredient consists of C3G. In other embodiments the active ingredient consists of ProC3G™. In other embodiments, the active ingredient consists of PCA. In yet other embodiments, the active ingredient consists of a mixture or C3G and PCA or consists of a mixture of ProC3G™ and PCA. In one embodiment, the PCA is present at about 97% of the total active ingredient.

In other embodiments the active ingredients are metabolites of anthocyanins or mixtures of metabolites. In certain embodiments, the active ingredient is a mixture of at least one anthocyanin and at least one of its metabolites. In certain embodiments the active ingredient is a mixture of an anthocyanidin and at least one of its metabolites. In certain embodiments the active ingredient is a mixture of at least one anthocyanin and at least one anthocyanidin. In certain embodiments the active ingredient is a mixture of at least one anthocyanin, at least one anthocyanidin, at least one metabolite of an anthocyanin and at least one metabolite of an anthocyanidin. In certain embodiments the active ingredient is a mixture of at least one anthocyanin and at least one metabolite of an anthocyanidin. In certain embodiments the active ingredient is a mixture of at least one anthocyanidin, at least one metabolite of an anthocyanin.

In some embodiments the active ingredient is a mixture of C3G and PCA.

In certain embodiments the method involves administration of no other anti-inflammatory medicines or no other therapeutic active ingredients.

The invention also provides methods of decreasing inflammation of a joint in a mammalian subject, the method comprising orally administering to the subject a composition comprising an active ingredient selected from the group consisting of an anthocyanin or a metabolite thereof, or an anthocyanidin or a metabolite thereof or a combination thereof, wherein said composition has the effect of decreasing levels of MMP1, TFG-beta, and MMP13 in the synovial fluid of the joint and increasing the levels of TIMP-1, VEGF, IL-10 and 11-4 in the synovial fluid of the joint to cause a reduction of inflammation in the joint.

The invention also provides methods of treating an injured or arthritic joint in a mammalian subject, the method comprising: orally administering to the subject a composition comprising an active ingredient selected from the group consisting of ProC3G™, cyanidin-3-glucoside (C3G) or protocatechuic acid (PCA) or a combination thereof, wherein said composition has the effect of increasing in the cartilage of the injured or arthritic joint expression levels of lubricin, aggrecan and collagen II to treat the injured or arthritic joint.

In addition, the invention provides a method of providing a chondro-nutritive and/or chondro-protective environment to the joint by the same oral administration discussed herein. By improving the anabolic/catabolic state, by decreasing negative proinflammatory cytokines, an increasing positive cytokines and factors, the synovial fluid, synovium and cartilage in a joint are primed for a better healing environment should an injury occur.

The invention provides methods of treating an injured or arthritic joint in a mammalian subject (to reduce inflammation and promote healing), the method comprising orally administering to the subject a composition comprising an active ingredient selected from the group consisting of ProC3G™, cyanidin-3-glucoside (C3G) or protocatechuic acid (PCA) or a combination thereof, wherein said composition has the effect of increasing in the cartilage of the injured or arthritic joint expression levels of lubricin, aggrecan and collagen II to treat the injured or arthritic joint.

The invention also provides methods of promoting healing of cartilage in a joint of a mammalian subject, the method comprising orally administering to the subject a composition comprising an active ingredient selected from the group consisting of ProC3G™, cyanidin-3-glucoside (C3G) or protocatechuic acid (PCA) or a combination thereof, wherein said composition has the effect of increasing in the cartilage expression levels of lubricin, aggrecan and collagen II to promote healing of the cartilage.

The invention also provides methods of decreasing levels of C reactive protein (CRP) in a joint of a mammalian subject, the method comprising orally administering to the subject a composition comprising an active ingredient selected from the group consisting of ProC3G™, cyanidin-3-glucoside (C3G) or protocatechuic acid (PCA) or a combination thereof, wherein said composition has the effect of decreasing levels of CRP in the plasma of the subject.

### EXAMPLES

### Example 1—Oral Ingestion Rabbit Study

The study was approved by the Institutional Animal Care and Use Committee (IACUC) of Thomas D. Morris, Inc. 28 New Zeeland White rabbits were selected for the study. Six such were randomly assigned to one of four groups; prophylactic cyanidin-3-glycoside (C3G)(referred to figures as CI 1-42), prophylactic protocatechuic acid (PCA)(referred to in figures as PCA 1-42), therapeutic C3G (referred to in figures as CI 42-70), and therapeutic PCA (referred to in figures as PCA 42-70). There were four controls, two each to the prophylactic and therapeutic groups. The controls did not receive any treatment.

The prophylactic groups were given PROC3G™ by Chromadex™ at a dose of 30 mg/kg of body weight of either C3G or PCA by mouth, 7 times per week for 42 days. The therapeutic groups' treatment was initiated on day 42 with a daily dose of PROC3G™ by Chromadex™ 7 times per week for 4 weeks. The dose of 30 mg of cyanidin-3-glucoside per kg of body weight calculates to be 0.177 millimoles per kg body weight. The dose of PCA was the same in terms of millimoles per kg body weight.

The experimental rabbits underwent surgery with an intent to create a severe degenerative arthritis by cutting the medial collateral ligament, the anterior cruciate ligament and removing the medial meniscus. A partial thickness laceration was made longitudinally on the lateral femoral condyle for future assessment of potential for repair. The surgery was performed by two licensed veterinarians. One surgeon was very experienced. The other surgeon was a recent graduate.

The magnitude of the arthritis was intentional in order to produce abundance of synovial reaction and synovial fluid for subsequent study. The experimental model was not designed for the likelihood of healing or cartilage repair due to the short time frame of the study.

At necropsy, the synovial fluid was harvested percutaneously and upon arthrotomy. The synovium was harvested from posterior compartment. The patella and adjacent synovium was procured as a separate specimen as was the medial and lateral condyle.

The synovial fluid and half of the synovium was frozen and sent to BioBoston Laboratory for chemical analysis. The other half of the synovium and bone was sent to McClinchery Histology in Stockbridge, Mich. for histological preparation and staining. Patellar slides were sent to BioBoston Contract Laboratories for histochemical staining and ICRS grading. Sample slides were examined for Lubrin at Myron Spector laboratory.

The cartilage status was subjected to a histological scale rating according to the International Cartilage Repair Society. Mainil-Varlet P, et al., The International Cartilage Repair Society (ICRS)—Histological Visual Scale. A preliminary Report of the Histological End Point Committee. I. Human Biopsies, Toronto Consensus. Europ Cells and Materials. Vol 4., Suppl. 1, 2002. (page 10).

Blood testing was performed prior to necropsy on C3G, PCA, glucose, MMP-3 and IGF-1 to assess any systemic effects.

Urine testing was performed prior to necropsy for C3G and PCA to assess the metabolic course. All reviews were performed by a blinded examiner.

### Results

There were 20 rabbits available for bilateral tissue harvest. Four had partial tissue available (#9, 2, 8, and 15). There was no tissue on three; #1, 20, 28. One of the controls (#27) for the therapeutic groups required early euthanasia due to illness. One of the PCA (#10) therapeutic group was lost due to illness. One of the C3G therapeutic group (#28) died in recovery and another died during the course of the study. Three others specimens were compromised by dislocated knee in one case and dislocated patella in two. The surgery in the compromised animals was performed by the recent graduate licensed veterinarian.

There were 40 specimens sent from 20 rabbits for bilateral chemical analysis. There were three unilateral specimens. There were no specimens sent for histology on four animals; 10 #16, 20, 22, 28. Partial specimens were sent on #10 and 15.

There was no evidence of healing of the lateral femoral condylar surgically induced partial thickness laceration within the short duration of the study.

Various assays were performed to determine levels of certain biomolecules associated with inflammation (e.g. proinflammatory cytokines), anabolic or catabolic processes (e.g. IGF-1, EGF, TIMP, MMP-3, MMP-1, Adamts-5). These tests results are provided below.

### MMP-3

MMP3 is known as matrix metallopeptidase 3, or stromelysin 1 or progelatinase. Proteins of the matrix metalloproteinase (MMP) family are involved in the breakdown of 20 extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases.

### Synovial Fluid—Detected by ELISA

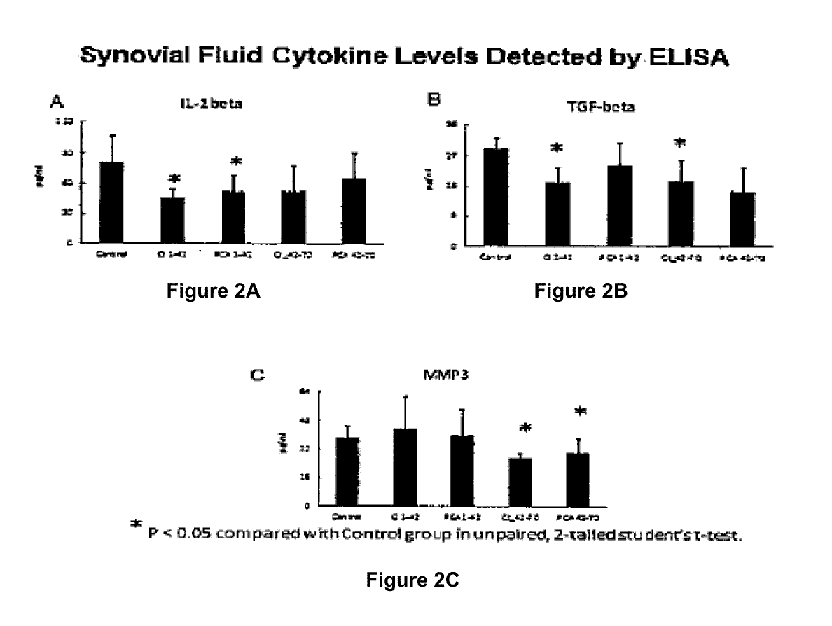

MMP3 was decreased in the therapeutic groups for C3G and PCA. See FIG. 2C.

### Synovial Tissue (Synovium)—Detected by ELISA

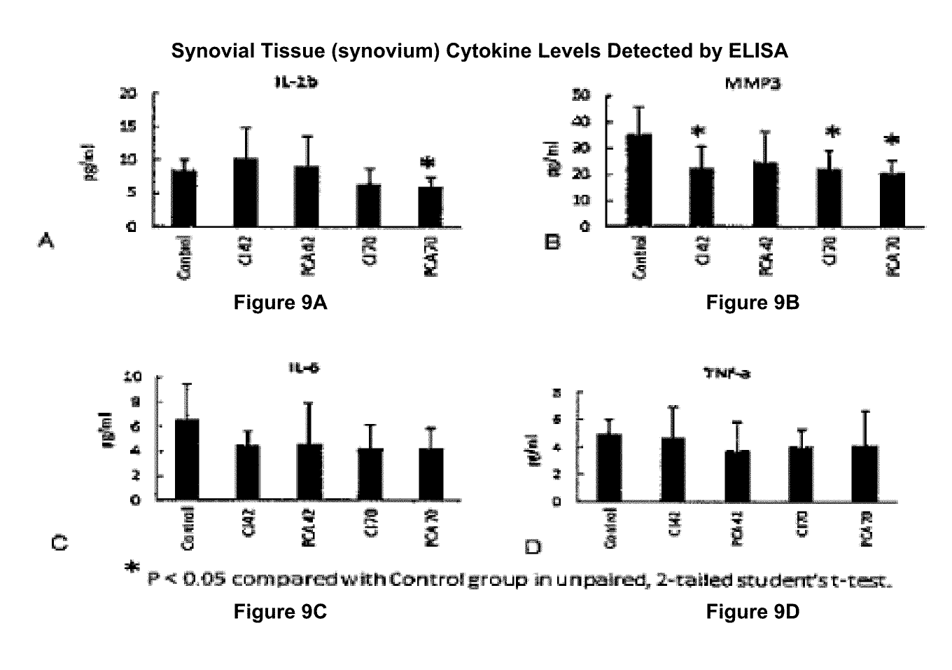

MMP-3 levels were reduced in all four groups. See FIG. 9B.

### Synovial Tissue (Synovium)—Gene Expression Level Detected by Real-Time PCR

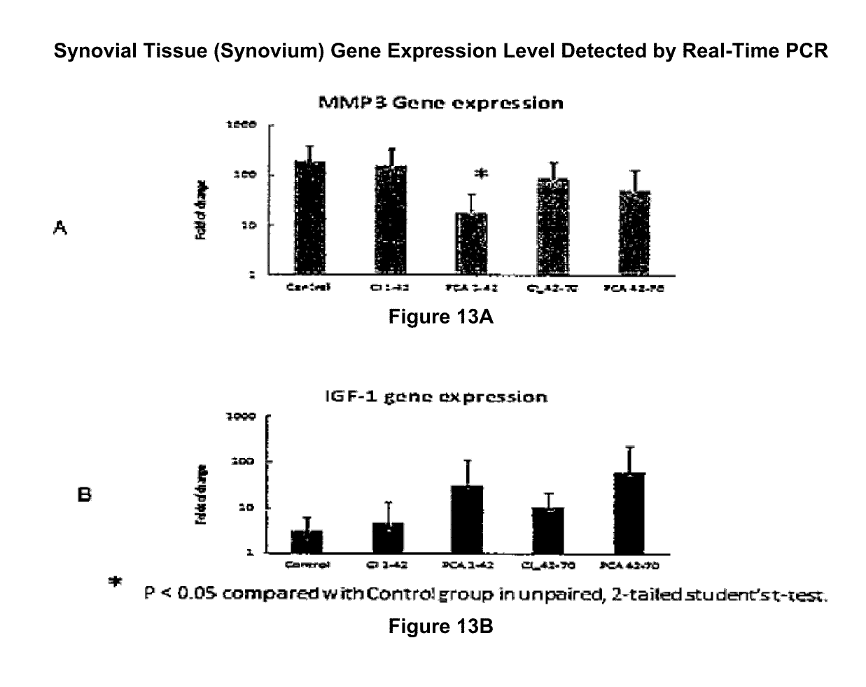

MMP-3 gene expression was decreased in all groups. See FIG. 13A.

### Histochemical Scoring Analysis

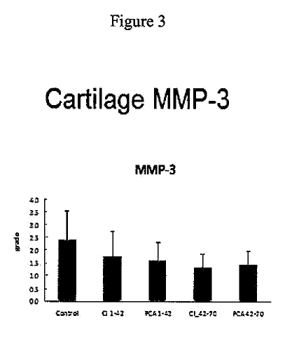

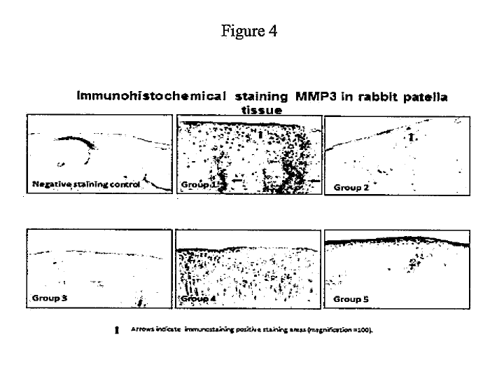

MMP-3 expression in cartilage was reduced in the both the therapeutic and the prophylactic G3G and PCA groups. See FIGS. 3 and 4.

### IGF-1

Insulin-like growth factor 1 (IGF-1), also called somatomedin C, is a protein plays an important role in childhood growth and continues to have anabolic effects in adults. Its primary action is mediated by binding to its specific receptor, the insulin-like growth factor 1 receptor (IGF1R), which is present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the anterior pituitary gland, is released into the blood stream, and then stimulates the liver to produce IGF-1. IGF-1 then stimulates systemic body growth, and has growth-promoting effects on almost every cell in the body, especially skeletal muscle, cartilage, bone, liver, kidney, nerves, skin, hematopoietic cell, and lungs.

IGF-1 was increased in the plasma only in the prophylactic PCA group as measured by PG/ML.

Control group: 25 PG/ML

Prophylactic C3G group: 15.8 PG/ML

Prophylactic PCA group: 43.0 PG/ML

Therapeutic C3G group: 28.5 PG/ML

Therapeutic PCA group: 29.0 PG/ML

### Synovial Fluid—Detected by ELISA

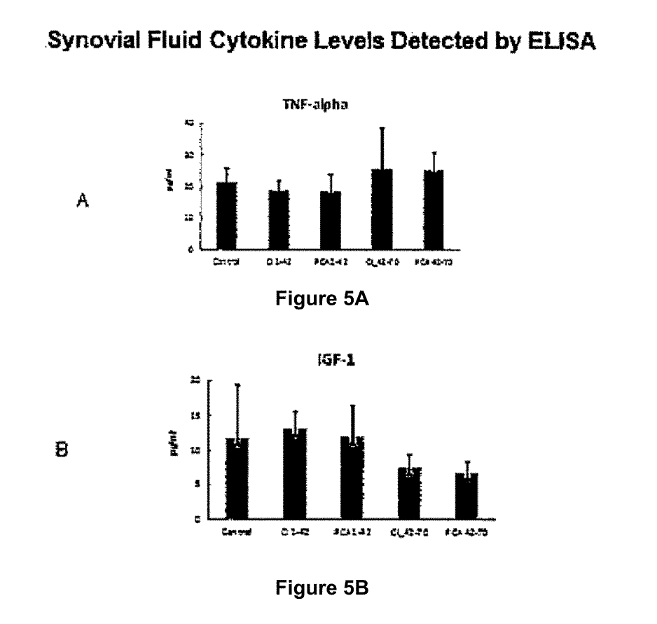

IFG-1 was not statistically significantly changed in all groups. See FIG. 5B.

### Synovial Tissue (Synovium)—Detected by ELISA

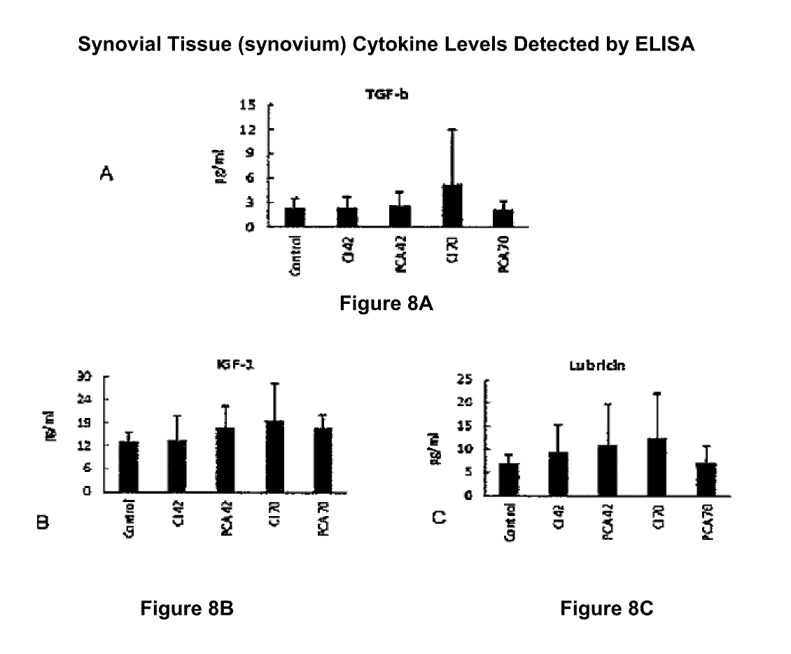

IGF-1 was increased in all groups. See FIG. 8B.

### Synovial Tissue (Synovium)—Gene Expression Level Detected by Real-Time PCR

IGF-1 was increased in all groups. See FIG. 13B.

### MMP-1

Matrix metalloproteinase-1 (MMP-1) also known as interstitial collagenase and fibroblast collagenase is an enzyme that is involved in the breakdown of extracellular matrix in normal physiological processes, such as embryonic development, reproduction, and tissue remodeling, as well as in disease processes, such as arthritis and metastasis. Specifically, MMP-1 breaks down the interstitial collagens, types I, II, and III.

### Synovial Fluid—Detected by ELISA

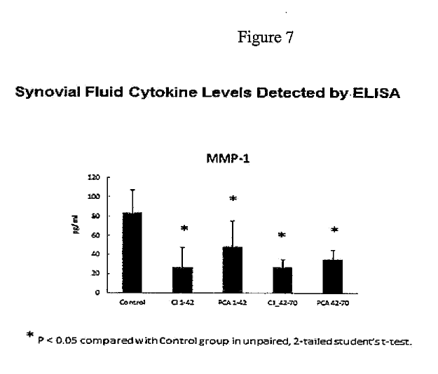

MMP-1 was decreased significantly in all groups. See FIG. 7.

### Synovial Tissue (Synovium)—Detected by ELISA

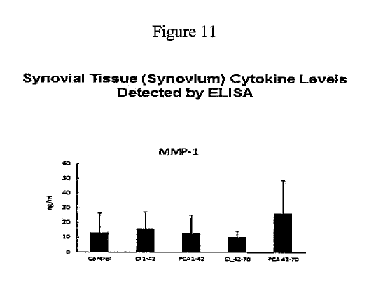

The therapeutic C3G group showed a decrease in MMP-1 in the synovium. See FIG. 11.

### Il-1 Beta

Interleukin-1 beta (IL-1β) also known as catabolin, is a cytokine protein. This cytokine is an important mediator of the inflammatory response, and is involved in a variety of cellular activities, including cell proliferation, differentiation, and apoptosis. Increased production of IL-1B causes a number of different auto-inflammatory syndromes.

### Synovial Fluid—Detected by ELISA

Levels of IL-1 beta were decreased in all groups. See FIG. 2A.

### Synovial Tissue (Synovium)—Detected by ELISA

Levels of IL-1 beta were decreased in the C3G and PCA therapeutic groups, but were increased in the two prophylactic groups. See FIG. 9A.

### Expression in Cartilage

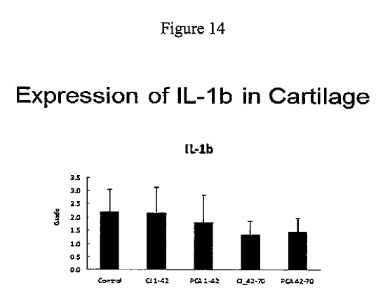

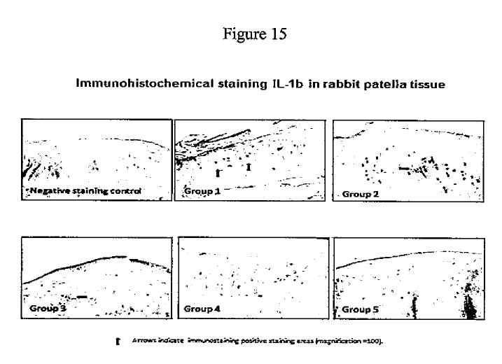

FIGS. 14 and 15 show that expression of IL-Beta in cartilage is decreased in all four groups.

### TGF-beta

TGF-β induces apoptosis in numerous cell types. Transforming growth factor beta (TGF-β) is a protein that controls proliferation, cellular differentiation, and other functions in most cells. It is a type of cytokine which plays a role in immunity, cancer, bronchial asthma, heart disease, diabetes, hereditary hemorrhagic telangiectasia, Marfan syndrome, vascular Ehlers-Danlos syndrome Loeys-Dietz syndrome, Parkinson's disease and AIDS.

### Synovial Fluid—Detected by ELISA

TGF-beta was decreased in all groups. See FIG. 2B.

### Synovial Tissue (Synovium)—Detected by ELISA

TGF-beta was increased in the C3G therapeutic group. See FIG. 8A.

### Lubricin

Proteoglycan 4 or lubricin is a proteoglycan that acts as a joint/boundary lubricant. Lubricin is present in synovial fluid and on the surface (superficial layer) of articular cartilage and therefore plays an important role in joint lubrication and synovial homeostasis.

Exposure of synoviocytes, chondrocytes and cartilage explants to proinflammatory cytokines such as Il-1 and TNF-alpha results in a marked reduction in the expression and/or abundance of secreted lubricin, with corresponding alterations in the amounts of cartilage-associated lubricin. Jones A R C, et al., European Cells and Materials Vol. 13, 2007 (pages 40-45) 2007.

Studies have shown that IL-1 inhibits the presence of lubricin and IGF-1 increases it synthesis. Flannery, C R, et al., Biochemical and Biophysical Research Communications. Vol. 254, Issue 3, 27 Jan. 1999, pages 535-541.

### Synovial Tissue (Synovium)—Detected by ELISA

Lubricin was increased in 3 of the 4 groups in the synovium (increase in both C3G groups and increase in the prophylactic PCA group)(see FIG. 8C).

### Expression in Cartilage

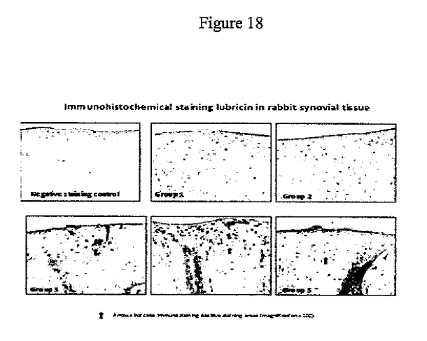

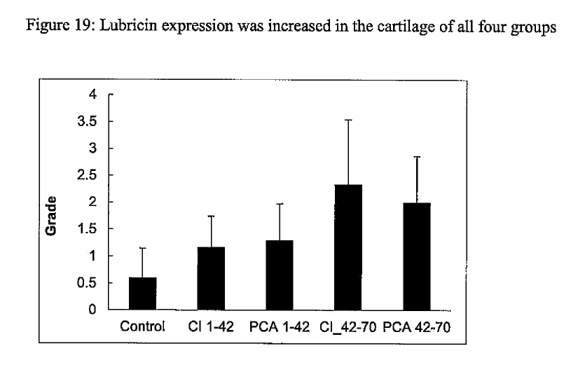

FIGS. 18 and 19 show that lubricin expression was increased in the cartilage of all four groups.

### Il-6

Interleukin 6 (IL-6) is an interleukin that acts as both a pro-inflammatory cytokine and an anti-inflammatory myokine. IL-6 is secreted by T cells and macrophages to stimulate immune response, e.g. during infection and after trauma, especially burns or other tissue damage leading to inflammation. In addition, osteoblasts secrete IL-6 to stimulate osteoclast formation. IL-6's role as an anti-inflammatory cytokine is mediated through its inhibitory effects on TNF-alpha and IL-1, and activation of IL-1ra and IL-10. IL-6 is an important mediator of fever and of the acute phase response.

### Synovial Tissue (Synovium)—Detected by ELISA

Il-6 was decreased in all groups. See FIG. 9C.

### TNF-Alpha

Tumor necrosis factor (TNF, cachexin, or cachectin, and formerly known as tumor necrosis factor alpha or TNFα) is an adipokine involved in systemic inflammation and is a member of a group of cytokines that stimulate the acute phase reaction. It is produced chiefly by activated macrophages, although it can be produced by many other cell types such as CD4+ lymphocytes, NK cells, neutrophils, mast cells, eosinophils, and neurons. The primary role of TNF is in the regulation of immune cells. TNF, being an endogenous pyrogen, is able to induce fever, apoptotic cell death, cachexia, inflammation and to inhibit tumorigenesis and viral replication and respond to sepsis via IL1 and IL6 producing cells.

TNF promotes the inflammatory response, which, in turn, causes many of the clinical problems associated with autoimmune disorders such as rheumatoid arthritis, ankylosing spondylitis, inflammatory bowel disease, psoriasis, hidradenitis suppurativa and refractory asthma.

TNF and Il-1 are considered master cytokines in chronic, destructive arthritis. In fact, therapeutic approaches to rheumatoid arthritis (RA) is mainly focuses on TNF. Analysis of cytokine patterns in early synovial biopsy specimens of RA patients reveals prominent TNF staining in 50% of the patients, whereas IL-1beta staining was evident in 100% of the patients. Van den Berg W B., Ann Rheum Dis., November 2000; 59 (Supp 1); i81-i84.

### Synovial Fluid—Detected by ELISA

TNF-alpha levels showed a slight decrease in both of the prophylactic groups. See FIG. 5A.

Synovial Tissue (Synovium)—detected by ELISA

TNF-alpha levels were decreased in all groups. See FIG. 9D.

### ADAMTS-5

ADAMTS-5 is a disintegrin and metalloproteinase with thrombospondin motifs. ADAMTS5 is a member of the ADAMTS protein family. Members of the family share several distinct protein modules, including a propeptide region, a metalloproteinase domain, a disintegrin-like domain, and a thrombospondin type 1 (TS) motif. Individual members of this family differ in the number of C-terminal TS motifs, and some have unique C-terminal domains. The enzyme encoded by this gene contains two C-terminal TS motifs and functions as aggrecanase to cleave aggrecan, which is a critical component of cartilage and joints. Thus ADAMTS-5 has a catabolic effect on cartilage.

### Synovial Fluid—Detected by ELISA

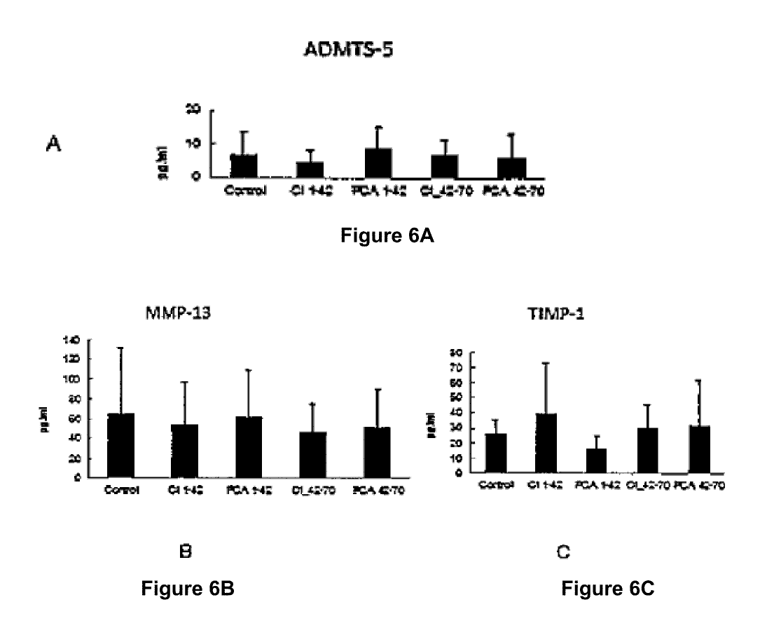

ADAMTS-5 was decreased in the both C3G groups and an in the therapeutic PCA group. See FIG. 6A.

### Synovial Tissue (Synovium)—Gene Expression Level Detected by Real-Time PCR

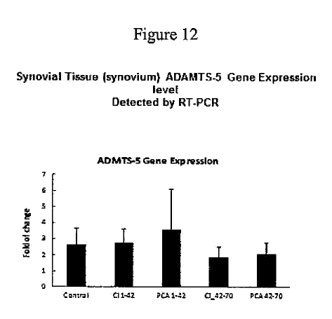

ADAMTS-5 gene expression was decreased in the synovium in the C3G and the PCA therapeutic groups. See FIG. 12.

### MMP-13

MMP-13 is also known as collagenase 3 and is an enzyme and a member of the matrix metalloproteinase (MMP) family. During embryonic development, MMP13 is expressed in the skeleton as required for restructuring the collagen matrix for bone mineralization. In pathological situations it is highly overexpressed; this occurs in human carcinomas, rheumatoid arthritis and osteoarthritis. Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. The protein encoded by this gene cleaves type II collagen more efficiently than types I and III. It may be involved in articular cartilage turnover and cartilage pathophysiology associated with osteoarthritis.

### Synovial Fluid—Detected by ELISA

MMP-13 was decreased in all groups. See FIG. 6B.

### Synovial Tissue (Synovium)—Detected by ELISA

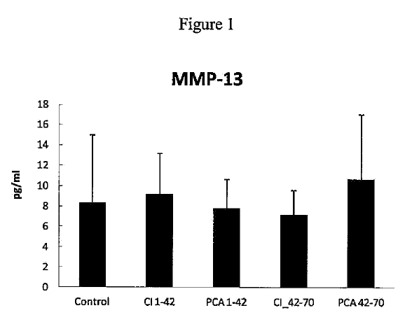

MMP-13 levels were slightly decreased in the C3G therapeutic group and very slightly decreased in the PCA prophylactic group. The levels seemed to be increased slightly in the PCA therapeutic and the C3G prophylactic group but this is not a statistical significant change. See FIG. 1.

### TIMP-1

TIMP metallopeptidase inhibitor 1, also known as TIMP1, and is a tissue inhibitor of metalloproteinases. It is a glycoprotein that is expressed from the several tissues of organisms. This glycoprotein is a natural inhibitor of the matrix metalloproteinases (MMPs), a group of peptidases involved in degradation of the extracellular matrix. In addition to its inhibitory role against most of the known MMPs, the encoded protein is able to promote cell proliferation in a wide range of cell types, and may also have an anti-apoptotic function.

### Synovial Fluid—Detected by ELISA

The levels were elevated in both C3G groups and in the PCA therapeutic group. See FIG. 6C.

Synovial Tissue (Synovium)—Gene Expression Level Detected by Real-Time PCR

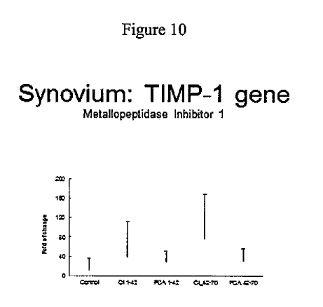

There was an increase in gene expression in the synovium in all four test groups. See FIG. 10.

### VEGF

VEGF is vascular endothelial growth factor that causes neovascularization and stops apoptosis of synovial cells.

### Synovium—Detected by ELISA

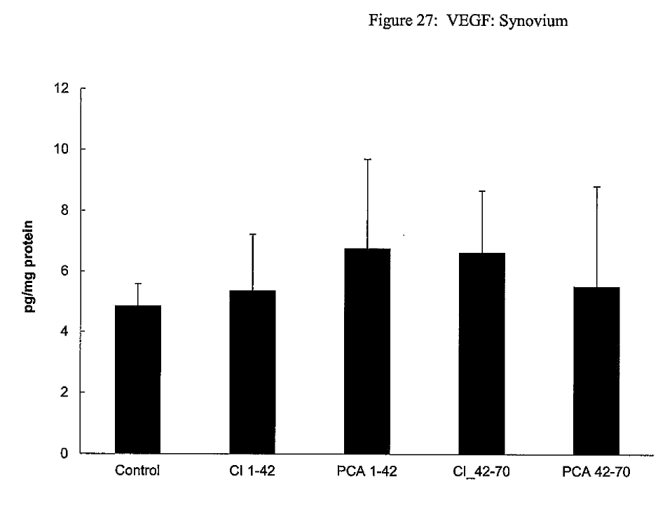

The levels of VEGF were increased in all four groups. See FIG. 27.

### IL-10

Interleukin-10 (IL-10), also known as human cytokine synthesis inhibitory factor (CSIF), is an anti-inflammatory cytokine. IL-10 is capable of inhibiting synthesis of pro-inflammatory cytokines such as IFN-γ, IL-2, IL-3, TNFα and GM-CSF made by cells such as macrophages and regulatory T-cells. It also displays a potent ability to suppress the antigen-presentation capacity of antigen presenting cells.

### Synovial Fluid—Detected by ELISA

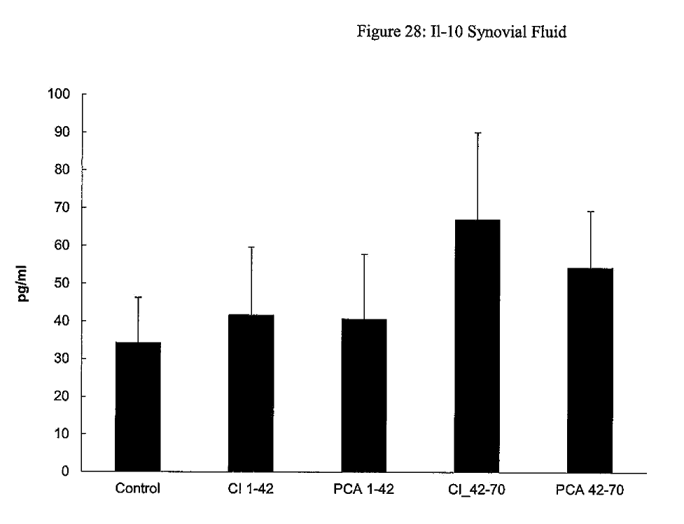

Levels of IL-10 were increased in all four groups. See FIG. 28.

### Il-4

The presence of IL-4 in extravascular tissues promotes alternative activation of macrophages into M2 cells and inhibits classical activation of macrophages into M1 cells. An increase in repair macrophages (M2) is coupled with secretion of IL-10 and TGF-β that result in a diminution of pathological inflammation.

### Synovial Fluid—Detected by ELISA

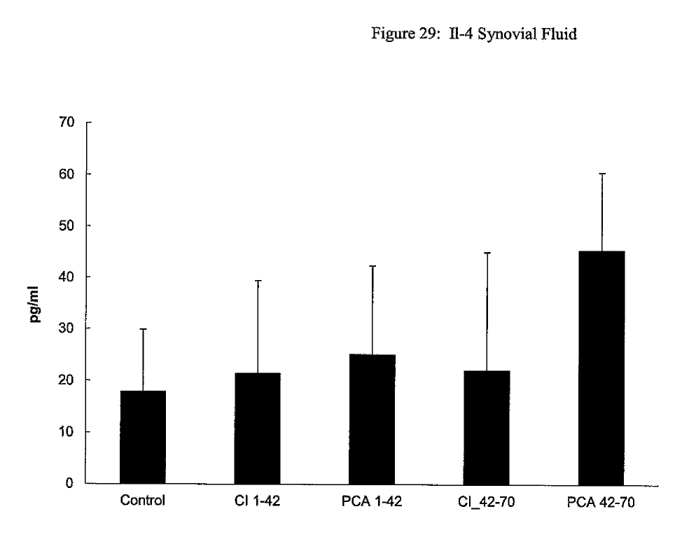

All four groups showed an increase in IL-4. See FIG. 29.

### Collagen II

Collagen II is the bases for articular cartilage and hyaline cartilage. It makes up 50% of all protein in cartilage and 85-905 of the collagen of articular cartilage.

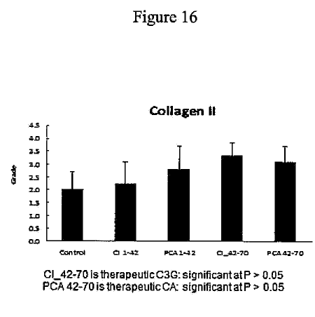

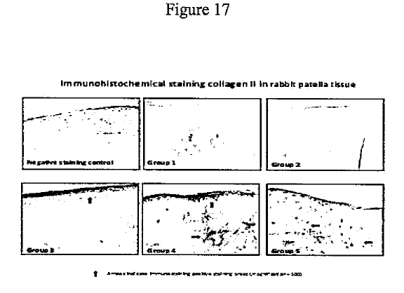

FIGS. 16 and 17 show that collagen II expression in cartilage is increased in all 4 groups.

### Aggrecan

Aggrecan is a major component of cartilage extracellular matrix and it imparts compressive resistance to the tissue.

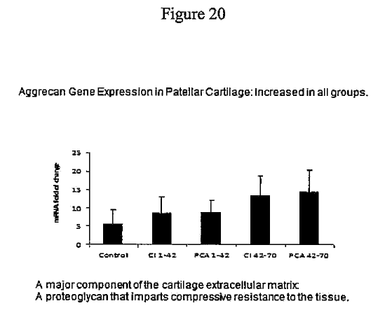

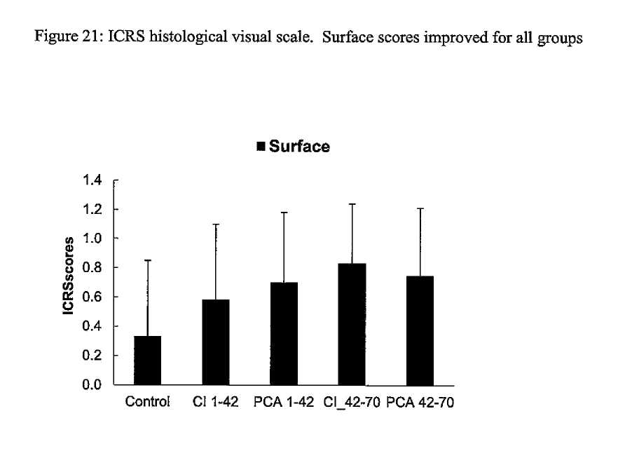

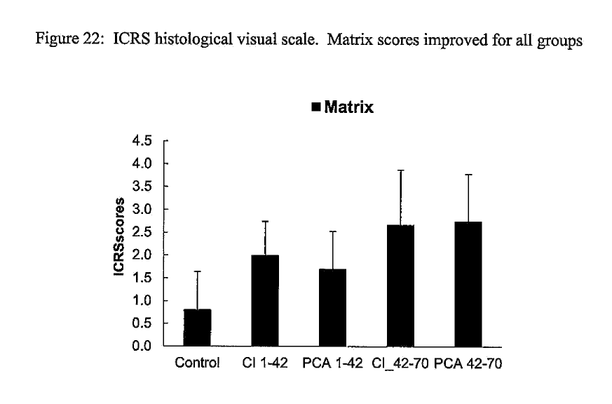

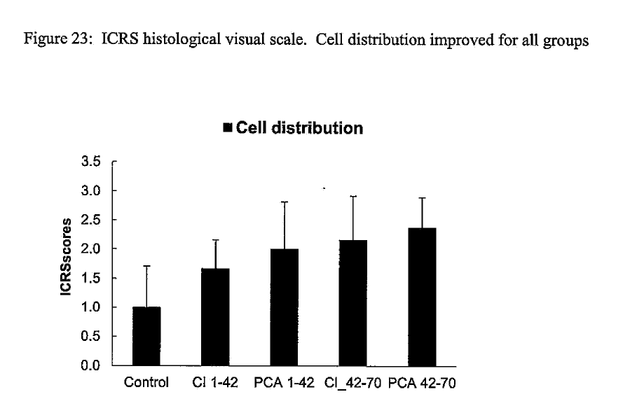

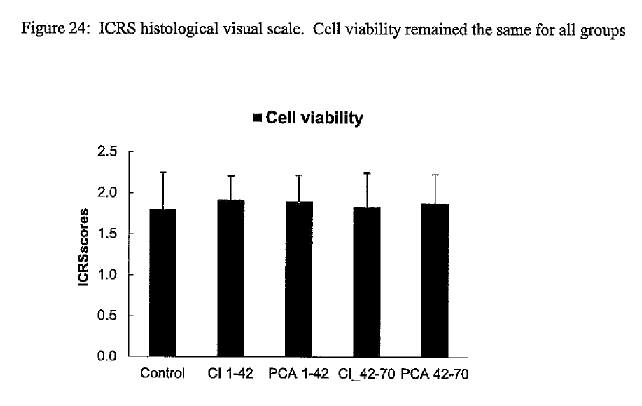

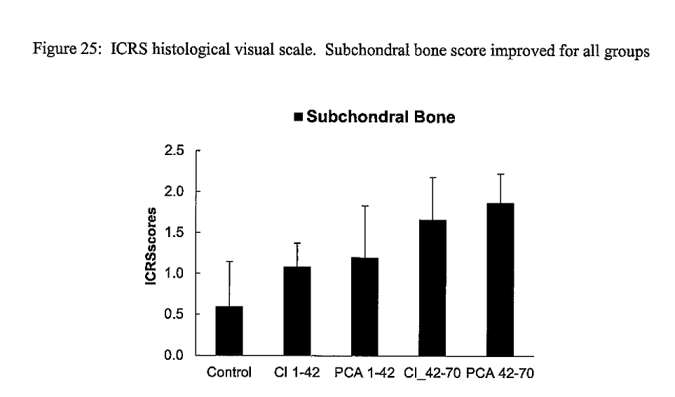

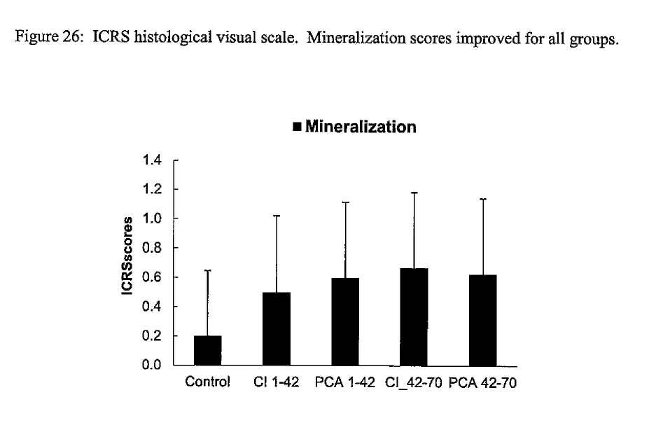

FIG. 20 shows increased expression of aggrecan in patellar cartilage of all four groups.

### White Blood Cell Studies—Anti-Inflammatory Effects

In addition to testing for the increase or decrease of various factors and cytokines, tests were run to count the number of white blood cells, including polymorphonuclear leukocytes (PMN), macrophages (Mac) and lymphocytes (Lym). Lymphocyte and macrophage cell numbers increase when there is inflammation and they are involved in the release of catabolic cytokines that are released into the joint.

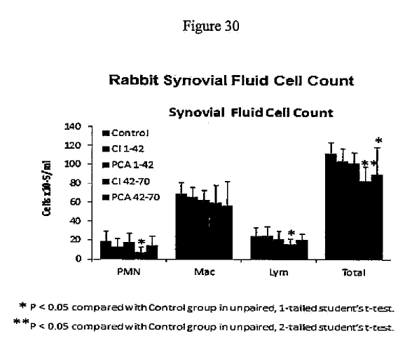

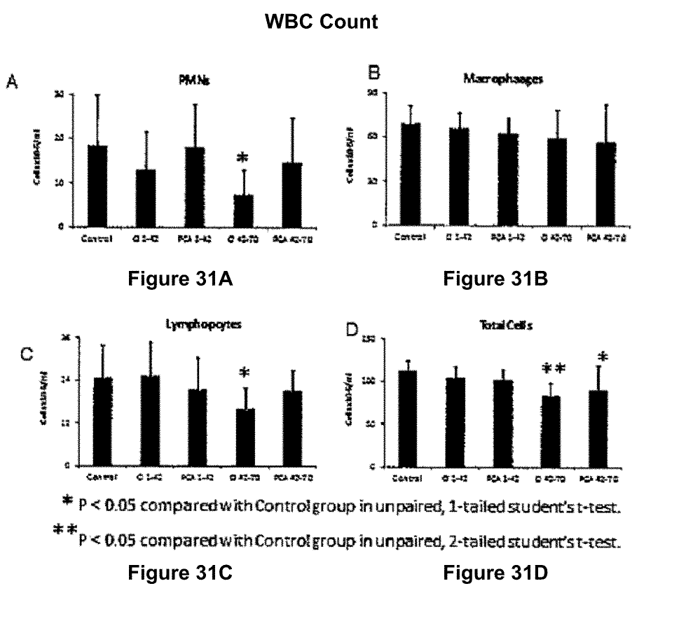

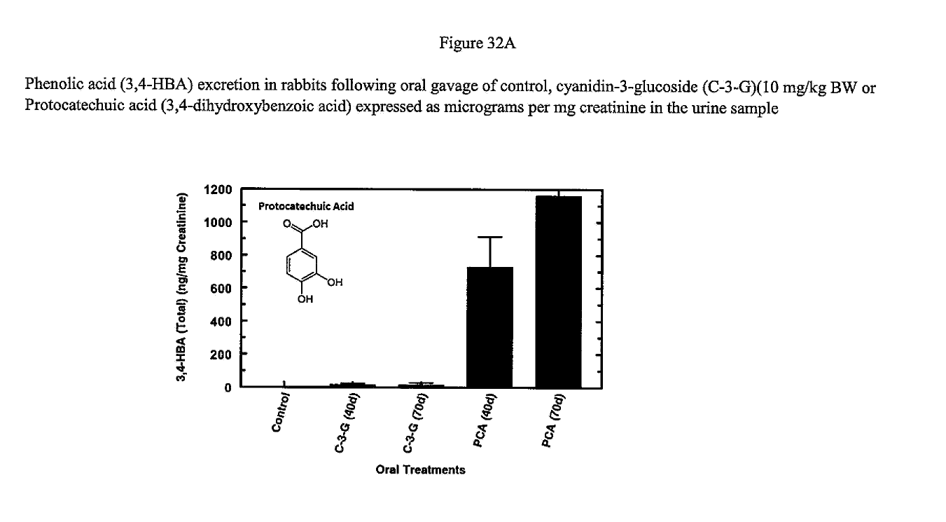

FIG. 30 and FIG. 31D show that the total white blood cell count in the synovial fluid was decreased in all 4 study groups as well as the numbers of macrophages was also decreased (see FIG. 31B). FIG. 31C shows that the number of lymphocytes in the synovial fluid was also decreased in 3 of the groups (both the PCA groups and the C3G therapeutic group). FIG. 31A shows that the number of PMN s was also reduced in the synovial fluid of all four groups, although most noticeable in the C3G groups.

### Discussion

The hypothesis was confirmed. There was significant reduction in the inflammation as measured by the white blood count and differential cell count. The synovium showed enhancement of the anabolic genes with a corresponding inhibition of the catabolic genes. The synovial fluid reflected the alteration with an increase in anabolic cytokines and a corresponding decrease in catabolic cytokines. The articular cartilage responded with histochemical and ICRS histological scoring evidence of enhanced nutrition and resultant protection of the patellar articular cartilage from the treatments. Medial or lateral compartment cartilage were not studied because they were directly affected by the surgical procedure and the patella was spared from the surgical intervention.

The dosage given was effective for both C3G and PCA. The regimen of five times per week proved practical and effective. There was an apparent benefit seen in the results of the 6 week duration of the prophylactic group versus the shorter 4 week duration in the therapeutic groups. This may indicate a longer duration has greater potential for intra-articular change.

The failure of healing of the partial thickness lateral femoral condylar laceration was not unexpected. The duration of the study was short. The space within a laceration is minimal so as not to house or hold blood or cells. In addition, there is constant shearing motion of the laceration as well as weight bearing forces. Such healing would likely require addition of cells in a blood clot with immobilization to reduce the shearing forces and a much longer period of time for restoration. It has been reported that fibrocartilage repair existed for as long as six years and conversion to hyaline cartilage was seen at 20 years. Johnson L L, Delano M C, Spector M, Jeng L, Pittsley A, Gottschalk A. The biological response following autogenous bone grafting for large volume defects of the Knee: index surgery through 12-21 year follow-up. Cartilage Volume 3 Issue 1. January 2012. Pp 85-98. First published on Aug. 16, 2011 as DOI: 10.117711947603511413568.

### Example 2: CRP Studies

C-reactive protein (CRP) is a protein found in the blood plasma, the levels of which rise in response to inflammation (i.e., C-reactive protein is an acute-phase protein). Its physiological role is to bind to phosphocholine expressed on the surface of dead or dying cells (and some types of bacteria) in order to activate the complement system via the C1Q complex. CRP rises within two hours of the onset of inflammation, up to a 50,000-fold, and peaks at 48 hours. Its half-life of 48 hours is constant, and therefore its level is determined by the rate of production and hence the severity of the precipitating cause. CRP is thus a screen for inflammation.

Knee surgery was performed to create a severe irreparable degenerative knee joint, which in turn elevated the CRP in the plasma to average of 8.8 mg/mL. A historical normal amount of CRP is 3.15 micrograms/mL. See Sun H, et al., Am J Pathol. October 2005; 167(4): 1139-1148. Rabbit CRP levels are close to the mean values of healthy middle-aged humans (2.82 mg/L) reported in the literature. See Ockene I S, et al., Clin Chem. 2001; 47:444-450.

The controls in the study were animals that had surgery and received no treatment. The prophylaxis test groups had the surgery and received 6 weeks of oral C-3-G or PCA 7 times per week as described in example 1. The therapeutic treatment group had surgery, received no treatment for 41 days and then at day 42 received oral C-3-G or PCA 7 times per week as described in example 1.

The CRP levels measured for the control group at 42 days (those receiving no treatment) showed an increase over normal levels: 8.8 mg/mL. The levels for the prophylaxis test group after treatment were reduced down to normal levels as follows:

Cyanidin-3-glucoside: CRP 3.1 micrograms/mL

Protocatechuic acid: CRP 3.6 micrograms/mL

This shows that the initiation of the treatment at time zero did not allow the CRP to be elevated, in fact the levels decreased.

The therapeutic group had the surgery, but received no treatment for 6 weeks and then received treatment for 4 weeks with same dosages of C3G or PCA that were used in the prophylaxis test groups. The plasma CRP levels the patients had very elevated levels. The plasma CRP was greatly elevated to 28.3 and 43 mg/mL (for the C3G and the PCA treatment groups, respectively). Thus, when the treatment was deferred for 6 weeks there was no effect on lowering the CRP, and in fact was elevated.

Thus, when C3G or PCA was given by oral route to a mammal immediately after injury, CRP, an indicator of inflammation was kept to normal levels (non-inflammatory levels).

What is claimed is:

1. A method of treating osteoarthritis in a patient having an osteoarthritic joint, comprising orally administering a therapeutically effective amount of PCA to the patient;
wherein the therapeutically effective amount elicits an osteoarthritis disease modifying response comprising:
decreased inflammation and an altered catabolic to anabolic state of the osteoarthritic joint and
improved chondronutrition and chondro-protection of articular cartilage of the osteoarthritic joint.

2. The method of claim 1, wherein the osteoarthritis disease modifying response further comprises:
increased IGF-1 expression and IGF-1 levels in a synovium and synovial fluid of the osteoarthritic joint,
a reduction of MMP3 expression and MMP3 levels in a synovium and cartilage of the osteoarthritic joint, and
increased expression levels of lubricin, aggrecan and type II collagen in a cartilage of the osteoarthritic joint.

3. The method of claim 1, wherein the osteoarthritis disease modifying response further comprises decreased levels of at least one of MMP1 and MMP13 in a synovial fluid of the osteoarthritic joint.

4. The method of claim 1, wherein the osteoarthritis disease modifying response further comprises increased levels of at least one of TIMP-1, VEGF, IL-10, and IL-4 in a synovial fluid of the osteoarthritic joint.

5. The method of claim 1, wherein the altered catabolic to anabolic state of the osteoarthritic joint comprises one or more of:
a reduction in levels of at least one of MMP3, MMP-1, MMP13, IL-1B, TNF-alpha, and ADAMTS-5 in a synovial fluid or synovium of the osteoarthritic joint;
an increase in levels of at least one of IGF-1, TIMP-1, VEGF, IL-10, and IL-4, in a synovial fluid or synovium of the osteoarthritic joint;
an increase in levels of lubricin in a synovium of the osteoarthritic join or an articular cartilage of the osteoarthritic joint;
an increase in aggrecan and collagen II in an articular cartilage matrix of the osteoarthritic joint.

6. The method of claim 1, wherein the therapeutically effective amount comprises administering an oral daily dose of 0.100 to 0.200 mmoles of PCA per kg of body weight of the patient.

7. The method of claim 1 wherein the therapeutically effective amount comprises administering a daily dose of the PCA for at least 6 weeks of a dose of 0.100 to 0.200 millimoles per kg body weight of the patient.

8. The method of claim 1, wherein the therapeutically effective amount comprises administering a daily dose of the PCA for at least 10 weeks of a dose of 0.100 to 0.200 millimoles per kg body weight of the patient.

9. The method of claim 1, wherein the therapeutically effective amount comprises administering a daily dose of the PCA for at least 4 weeks of a dose of 0.100 to 0.200 millimoles per kg body weight of the patient, starting at day 42 after an injury of a joint or surgery of a joint.

10. The method of claim 5, wherein increasing expression levels of a lubricin, aggrecan and type II collagen in the cartilage of an osteoarthritic joint, results in a chondro-protective and/or chondro-nutritive effect on the synovial joint.

11. The method of claim 1, wherein the PCA is provided as a prophylactic treatment to the patient in advance of a surgery to the osteoarthritic joint.

12. The method of claim 1, further comprising providing PCA to the patient after a surgery.

13. A dosing regimen for modifying an osteoarthritic disease process in a patient having an osteoarthritic joint, wherein the dosing regimen comprises an oral administration of 0.100 to 0.200 mmoles of PCA per kg of body weight of PCA to the patient, thereby modifying the osteoarthritic disease process.

14. The dosing regimen of claim 13, wherein modifying the osteoarthritic disease process comprises one or more of:
decreased inflammation and an altered catabolic to anabolicstate of the osteoarthritic joint, thereby restoring a synovial cytokine homeostasis;
improved chondronutrition and chondro-protection of articular cartilage of the osteoarthritic joint;
decreased levels of MMP1 and MMP13 in a synovial fluid of the osteoarthritic joint;
increased IGF-1 expression and IGF-1 levels in a synovium and synovial fluid of the osteoarthritic joint;
a reduction of MMP3 expression and MMP3 levels in a synovium and cartilage of the osteoarthritic joint;
increased expression levels of lubricin, aggrecan and type II collagen in a cartilage of the osteoarthritic joint;
increased levels of TIMP-1, VEGF, IL-10, and IL-4 in the synovial fluid of the osteoarthritic joint;
a reduction in the levels of MMP3, MMP-1, MMP13, IL-1B, TNF-alpha, ADAMTS-5 in a synovial fluid or synovium of the osteoarthritic joint;
an increase in levels of IGF-1, TIMP-1, VEGF, IL-10, and IL-4 in the synovial fluid or synovium of the osteoarthritic joint;
an increase in levels of lubricin in a synovium of the osteoarthritic join or an articular cartilage of the osteoarthritic joint;
an increase in aggrecan and collagen II in an articular cartilage matrix of the osteoarthritic joint.

15. The dosing regimen of claim 13, wherein 0.100 to 0.200 millimoles per kg body weight of PCA is administered as a daily dose for at least 6 weeks.

16. The dosing regimen of claim 13, wherein 0.100 to 0.200 millimoles per kg body weight of PCA is administered as a daily dose for at least 10 weeks.

17. The dosing regimen of claim 13, wherein 0.100 to 0.200 millimoles per kg body weight of PCA is administered as a daily dose for at least 4 weeks starting at day 42 after an injury of a joint or surgery of an osteoarthritic joint.

18. A disease modifying osteoarthritic composition comprising PCA and one or more pharmaceutically acceptable carriers; wherein the PCA is at least 97% pure; and wherein the composition contains no additional therapeutic active ingredients.

19. The composition of claim 18, wherein the composition is formulated for an administration of an oral daily dose of 0.100 to 0.200 mmoles of PCA per kg of body weight of a patient.

20. The composition of claim 18, wherein the composition elicits an osteoarthritis disease modifying response comprising one or more of:
decreased inflammation and an altered catabolic to anabolic state of the osteoarthritic joint;
improved chondronutrition and chondro-protection of articular cartilage of an osteoarthritic joint;
decreased levels of MMP1 and MMP13 in a synovial fluid of an osteoarthritic joint;
increased IGF-1 expression and IGF-1 levels in a synovium and synovial fluid of the osteoarthritic joint;
a reduction of MMP3 expression and MMP3 levels in a synovium and cartilage of the osteoarthritic joint;
increased expression levels of lubricin, aggrecan and type II collagen in a cartilage of an osteoarthritic joint;
increased levels of TIMP-1, VEGF, IL-10, and IL-4 in the synovial fluid of an osteoarthritic joint;
a reduction in the levels of MMP3, MMP-1, MMP13, IL-1B, TNF-alpha, ADAMTS-5 in a synovial fluid or synovium of the osteoarthritic joint;
an increase in levels of IGF-1, TIMP-1, VEGF, IL-10, and IL-4 in the synovial fluid or synovium of an osteoarthritic joint;
an increase in levels of lubricin in a synovium of the osteoarthritic join or an articular cartilage of the osteoarthritic joint; and
an increase in aggrecan and collagen II in an articular cartilage matrix of the osteoarthritic joint.
